(19) United States
(12) Patent Application Publication
(10) Pub. No.: US 2010/0119493 A1
(43) Pub. Date: May 13, 2010

(54) Telencephalic Glial-Restricted Cell Populations and Related Compositions and Methods

(75) Inventors: Margot Mayer-Proschel, Pittsford, NY (US); Frederick G. Strathmann, IV, Bellevue, WA (US)

Correspondence Address: FISH & RICHARDSON P.C., P.O BOX 1022, Minneapolis, MN, 55440-1022, US

(73) Assignee: UNIVERSITY OF ROCHESTER, Rochester, NY (US)

(21) Appl. No.: 12/596,223

(22) PCT Filed: Apr. 16, 2008
(86) PCT No.: PCT/US08/60477

§ 371 Date: Jan. 14, 2010

Related U.S. Application Data
(60) Provisional application No. 60/912,387, filed on Apr. 17, 2007.

(51) Int. Cl. A61K 35/12; C12N 5/00

(52) U.S. Cl. 424/93.7; 435/325

(57) ABSTRACT

Provided herein are telencephalic glial-restricted precursor cell populations and related compositions. Related compositions include, but are not limited to, any cell or cell population derived from a population of telencephalic glial-restricted precursor cells. Further provided are methods of using and producing telencephalic glial-restricted precursor cell populations and related compounds.

### CROSS-REFERENCE TO RELATED APPLICATIONS

This application claims priority to U.S. Provisional Application No. 60/912,387, filed Apr. 17, 2007, which is incorporated herein by reference in its entirety.

### STATEMENT REGARDING FEDERALLY FUNDED RESEARCH

This invention was made with government support under Grant Nos. NS042800251 and 1T32NS051152-01 awarded by the National Institutes of Health. The government has certain rights in the invention.

### BACKGROUND

Injury to the central nervous system (CNS) is associated with multiple types of damage, all of which pose substantial challenges to tissue repair.

### SUMMARY

Provided herein are telencephalic glial-restricted precursor cell populations and related compositions. Further provided are methods of using and producing telencephalic glial-restricted precursor cell populations and related compounds. For example, the disclosed methods include methods of treating a CNS lesion in a subject comprising administering telencephalic glial-restricted precursor cells, or cells derived from a telencephalic glial-restricted precursor cell, to the subject.

### DESCRIPTION OF DRAWINGS

The accompanying drawings, which are incorporated in and constitute a part of this specification, illustrate several of the disclosed methods and compositions and together with the description, serve to explain the principles of the disclosed methods and compositions.

FIGS. 1A, 1B, 1C and 1D are micrographs showing A2B5+ cells in the telencephalon. FIG. 1A shows A2B5⁺ cells in coronal sections of the developing striatum and dorsolateral neocortex of the E15 telencephalon. FIG. 1B shows that A2B5⁺ cells are absent in the developing hippocampal region. FIGS. 1C and 1D show that the dorsal A2B5⁺ region is not Olig2⁺ (FIG. 1C) while the ventral A2B5⁺ region partially overlaps with the Olig2⁺ domain in the developing striatum (FIG. 1D). FIG. 1E shows FACS data of A2B5⁺/PSA-NCAM⁻ stained cells shows three cell populations, including PSA-NCAM⁺, A2B5⁺/PSA-NCAM⁺, and A2B5⁺. Scale bar, 100 μm.

FIGS. 2A, 2B, and 2C are micrographs showing a subset of A2B5+ cells are also beta III tubulin+ in the E15 dorsal telencephalon. FIGS. 2A-C show the isolated A2B5⁺/PSA-NCAM⁻ cell population from the dorsal telencephalon included a beta III tubulin⁺ population, seen at 1 hour (FIG. 2A), 12 hours (FIG. 2B), and 4 days (FIG. 2C) post isolation. FIG. 2D is a histogram showing isolated A2B5⁺/PSA-NCAM⁻ cells stained and analyzed for beta III tubulin presence between E13 and E20. E15 was determined to be the peak time to isolate A2B5⁺/PSA-NCAM⁻/beta III tubulin⁻ cells as 21% of the E15 A2B5+/PSA-NCAM− population was beta III tubulin⁻. DAPI nuclear stain. Scale bars, 100 μm.

FIGS. 3A, 3B and 3C show an outline of the isolation procedure used to characterize the putative glial restricted precursor population. A2B5⁺/PSA-NCAM⁻ cells were selected by MACS resulting in a heterogeneous mixture of cells. For mass culture studies (FIG. 3A) and clonal analysis (FIG. 3B), cells were maintained in culture for two cell passages to select for proliferative cells and to remove the A2B5⁺ neuronal population. The resultant putative glial restricted precursor population was then plated at mass culture or clonal density and exposed to differentiating conditions including a pro-oligodendrocytic condition, a pro-astrocytic condition, or a proneuronal condition. Alternatively, the heterogeneous mixture of cells obtained from the MACS selection was plated at clonal density, and resultant clones were selectively passaged and split into the differentiation conditions (FIG. 3C).

FIGS. 4A, 4B, 4C, 4D, 4E and 4F are micrographs showing that the putative dorsal glial restricted precursor population can generate macroglial subtypes in mass culture. Putative glial restricted precursor cells generate GalC+ cells (FIG. 4A) and GFAP+ cells (FIG. 4C) but do not generate neurons (FIG. 4D) after 6 days of exposure to the appropriate differentiation conditions. FIG. 4B shows that after 4 days of growth in the pro-oligodendrocyte condition, O4+ cells were readily identifiable. FIGS. 4E and 4F show exposure of the putative glial restricted precursor population to BMP-4 is insufficient to result in detection of the known astrocyte marker GFAP until 10 days (FIG. 4E), but does induce the astrocyte precursor cell marker, CD44, after 6 days (FIG. 4F). DAPI nuclear stain (FIGS. 4D and 4F). Scale bars, 100 μm.

FIGS. 5A and 5B show photomicrographs of neuron generation from E15 unsorted dorsal and ventral telencephalic cells. In order to validate the pro-neuronal condition used, cells present in the E15 dorsal (FIG. 5A) and ventral (FIG. 5B) telencephalon before MACS selection were exposed to the pro-neuronal condition used for glial restricted precursor characterization and were found to generate beta III tubulin⁺ cells after 6 days in culture. Scale bars, 100 μm.

FIGS. 6A, 6B and 6C are micrographs showing clonal analysis of the putative dorsal glial restricted precursor further indicates glial restriction. To distinguish between the potential presence of an APC/OPC cell mixture and the presence of a glial restricted precursor population, the putative glial restricted precursor population was grown at clonal density and exposed to the differentiating conditions, resulting in the detection of clones containing GalC⁺ cells (FIG. 6A) clones containing GFAP⁺ cells (FIG. 6B) but no neuron containing clones (FIG. 6C). DAPI nuclear stain. Scale bars, 100 μm.

FIGS. 7A, 7B and 7C are micrographs showing clone splitting confirming the ability of the putative glial restricted precursor cell to generate both oligodendrocytes and astrocytes. Split clones of A2B5+/PSA-NCAM− founder cells can generate GalC⁺ cells (FIG. 7A) GFAP cells (FIG. 7B) but not neurons (FIG. 7C) and allows for the classification of the A2B5+/PSA-NCAM-Theta III tubulin⁻ cell as a glial restricted precursor cell. DAPI nuclear stain. Scale bars, 100 μm.

FIGS. 8A, 8B, 8C, 8D, 8E, 8F, 8G, 8H, and 81 are micrographs showing the dorsal telencephalon has the potential to generate glial restricted precursor cells independent of ventral cell infiltration. FIGS. 8A, 8B and 8C show that cells with the similar antigenic profile described for the dorsal glial restricted precursor population were isolated from two day in vitro grown dorsal explants, and can generate GalC⁺ cells (FIG. 8A) GFAP⁺ cells (FIG. 8B) but not neurons (FIG. 8C) in mass culture. FIGS. 8D, 8E and 8F show explant derived putative glial restricted precursors can generate clones containing GalC⁺ cells (FIG. 8D) clones containing GFAP⁺ cells (FIG. 8E) but no clones containing neurons (FIG. 8F) when exposed to the differentiation conditions. FIGS. 8G, 8H and 8I show split clones of explant derived putative glial restricted precursor founder cells can generate GalC⁺ cells (FIG. 8G) GFAP⁺ cells (FIG. 8I) but not neurons (FIG. 8I). DAPI nuclear stain. Scale bars, 100 μm.

FIGS. 9A, 9B, 9C, 9D, 9E, 9F, 9G, 9H, 9I and 9J are micrographs showing a glial restricted precursor population cell can be isolated from the E15 ventral telencephalon. FIGS. 9A, 9B and 9D show putative glial restricted precursor cells sharing the similar antigenic profile of the dorsal glial restricted precursor population were isolated from the E15 ventral telencephalon, consisting of the AEP and MGE. This cell population generated GalC⁺ cells (FIG. 9A) and GFAP⁺ cells (FIG. 9B) but not neurons (FIG. 9D) in mass culture. FIG. 9C shows putative glial restricted precursor cells do not make A2B5⁺/GFAP⁺ Type-II astrocytes in response to CNTF. To distinguish between APC/OPC presence and glial restricted precursor presence, ventral putative glial restricted precursor cells were grown at clonal density and generated GalC⁺ cells (FIG. 9E) and GFAP⁺ cells (FIG. 9F) but not neurons (FIG. 9G) when examined at the clonal level. Split clones of ventral putative glial restricted precursor founder cells generated GalC⁺ cells (FIG. 9H) and GFAP⁺ cells (FIG. 9I) but not neurons (FIG. 9J). DAPI nuclear stain, (FIGS. 9A, 9C-9J). Scale bars, 100 μm.

FIG. 10 is a histogram showing a summary of the generated clones from dorsal, ventral, and explant derived glial restricted precursor, with no significant difference (p>0.05; Student's t-test) between astrocyte and oligodendrocyte containing clone numbers.

FIGS. 11A, 11A′, 11B, 11B′, 11C, 11C′ are electronmicrographs and 11D, 11E, 11F, 11G, 11H and 11I are fluorescent micrographs, showing dorsal glial restricted precursors and explant derived dorsal glial restricted precursors produce compact myelin, in addition to the ability of both ventral and dorsal glial restricted precursors to make astrocytes in vivo. FIGS. 11A-C′ show EM images from the contralateral hemisphere of the transplanted shiverer forebrains showed a lack of dense, compacted myelin, consistent with the shiverer mutant phenotype, on longitudinally sectioned (FIG. 11A) and cross-sectioned (FIG. 11A′) neuronal fibers. The dorsal glial restricted precursor isolated from the E15 dorsal telencephalon and transplanted into the postnatal day 18 (P18) shiverer forebrain is capable of myelin formation as seen in longitudinally sectioned (FIG. 11B) and cross-sectioned (FIG. 11B′) neuronal fibers. Transplantation of the dorsal glial restricted precursor cell derived from two day in vitro grown E13 dorsal telencephalic explants into the P18 shiverer mutant forebrain produces compacted myelin as seen in longitudinally sectioned (FIG. 11C) and cross-sectioned (FIG. 11C′) neuronal fibers. FIGS. 11D-F show hPAP⁺ dorsal glial restricted precursors transplanted into the forebrains of PO rat pups generate hPAP⁺/GFAP⁺ cells after 10 days, as well as Olig2⁺ oligodendroglial cells (FIGS. 11G-I). DAPI nuclear stain (FIG. 11F). Scale bars for 11A-C′ as indicated, scale bars for 11D-I, 100 μm.

FIG. 12 shows a model for the generation of glial subtypes through telencephalic Glial Restricted Precursor (tGRP) populations. The dorsal telencephalon and ventral telencephalon give rise to glial restricted precursor populations with a primary developmental fate towards astrocyte and OPC generation, respectively. The classification of these two populations as true tGRP populations uses their isolation and in vitro characterization in order to remove the normal developmental cues promoting dorsal astrocyte generation and ventral OPC formation. As the ventral and dorsal telencephalon continues through development, each tGRP population has the potential to participate in a secondary developmental fate towards astrocytes ventrally, or OPCs dorsally. The developmental plasticity of each population is revealed in vitro and demonstrates the potential for oligodendrocyte and astrocyte development from a common precursor cell type.

FIG. 13A is a micrograph showing spinal cord GDA^{gp130} (CNTF induced) astrocytes express both GFAP and Olig2. Cells were grown for 4 days in the presence of growth factors.

FIG. 13B is a micrograph showing CNTF induced GFAP⁺ astrocytes derived from tGRPs do not resemble scGDA^{gbp130} based on a lack of Olig2/GFAP colocalization.

FIG. 14 shows intracellular redox status of ventral and dorsal tGRPs. As measured by the geometric mean of oxidized dye fluorescence, dorsal tGRPs have a higher intracellular redox level when compared to ventral tGRPs.

FIG. 15 A, B and C, are micrographs showing an indication that tGRPs generate GalC+oligodendrocytes via a PSA-NCAM/PDGFRalpha/Olig2+ intermediate. The passage of a tGRP through a classically described OPC (PSA-NCAM/PDGFRalpha/Olig2+) intermediate provides evidence that tGRps are responsible for the generation of OPCs in vivo and adds to the number of possible intermediate cell fates that are achievable with the use of tGRPs as a starting population.

### DETAILED DESCRIPTION

This disclosure is related to lineage restricted glial precursor cells from the telencephalon. For example, provided herein are telencephalic glial-restricted precursor (tGRP) cell populations. Related compositions are also provided and include, but are not limited to, any cell or cell population derived from a population of telencephalic glial-restricted precursor cells. An example of a related composition is a type-1 astrocyte, or population thereof, derived from a telencephalic glial-restricted precursor cell. Related compositions can also include other compounds, agents or molecules in combination with a tGRP cell or population, or a cell or cell population derived from a tGRP cell or cell population. Also provided are Olig2⁻ glial restricted precursor (GRP) cells and cell populations. Optionally, the Olig2⁻ GRPs are isolated from the dorsal telencephalon.

Further provided are methods of using and producing telencephalic glial-restricted precursor cell populations and related compositions. These methods include, but are not limited to, treating a CNS lesion in a subject comprising administering telencephalic glial-restricted precursor cells, or cells derived from a telencephalic glial-restricted precursor cell, to the subject. The cells can be administered in combination with other compounds, agents or molecules as described herein.

Telencephalic glial-restricted precursor cell populations include precursor populations in the ventral and dorsal telencephalon that generate astrocytes and oligodendrocytes. The dorsal glial precursor cells can be generated de novo from the dorsal telencephalon and they can be used for in vivo production of both myelin-forming oligodendrocytes and astrocytes upon transplantation into a subject.

Within the central nervous system (CNS), the greatest progress in identifying the specific cell populations involved in development has been achieved in the spinal cord. In the rat spinal cord, embryonic day 10.5 (E10.5) cells have been shown to represent a homogenous population of multipotent neuroepithelial stem cells (NEPs) capable of generating cells of both the neuronal and glial lineage.

Differentiated cell types arise from these NEP cells by way of lineage restricted intermediate precursor populations capable of extended proliferation and the generation of neurons or glia. The cells comprising the earliest intermediate precursor population restricted to oligodendrocyte and astrocyte formation, called glial restricted precursor cells (GRPs), can be isolated from the embryonic spinal cord as early as E12. Their ability to generate two antigenically distinct populations of astrocytes and oligodendrocytes has been established both in vitro and in vivo.

GRP cells are identified with the A2B5 antibody and do not express the Polysialylated form of Neural Cell Adhesion Molecule (PSA-NCAM). Freshly isolated GRP cells depend on basic fibroblast growth factor (bFGF) for survival and proliferation but, unlike oligodendrocyte progenitor cells (OPCs), are not defined by the expression of platelet-derived growth factor receptor-alpha (PDGFR-alpha) or Olig2. The OPC has been shown in vivo to arise at a later time point than the GRP, and the generation of oligodendrocytes from a GRP population has been demonstrated in vitro to occur through an OPC intermediate stage.

Additional characteristics distinguishing GRP cells from OPCs are the ability of the GRP cells to generate two types of astrocytes (that have been designated type-1 and type-2) in vitro and to generate both oligodendrocytes and astrocytes in vivo. Both type-1 and type-2 astrocytes are GFAP⁺, but only type-2 astrocytes co-label with the A2B5 antibody. Type-1 astrocytes are thought to arise from GRP cells through intermediate astrocyte progenitor cells (APC), while Type-2 astrocytes can require prior generation of OPCs as an intermediate step. Unlike OPCs, GRP cells readily generate astrocytes following transplantation into the adult CNS, while primary OPCs only generate oligodendrocytes in such transplantations.

The identification of GRP cells in the spinal cord gave rise to a generalized model of gliogenesis. This model of gliogenesis involves the progression from a multipotential NEP cell to a lineage restricted multipotent precursor cell population (e.g. GRPs) that in turn give rise to more restricted glial precursor cell types (e.g. OPCs and possibly APCs) and the eventual mature glial cells of the CNS (e.g. oligodendrocytes and astrocytes).

It has been ascertained through genetic and clonal in vitro experiments that a subset of cells from ventral regions of the telencephalon differentiate into PDGFR-alpha+ and/or Olig2+ oligodendrocyte progenitors, migrate away from their ventral origin, and give rise to mature oligodendrocytes throughout the brain. It appears that these cells express Olig1/2 to be fated towards oligodendrocytes as compound disruption of Olig1 and Olig2 results in a complete loss of oligodendrocytes.

Provided herein are telencephalic precursor cell populations capable of generating oligodendrocytes and astrocytes but that are unable to generate neurons under conditions that generally promote neuronal lineage. Examples of conditions that generally promote neuronal lineage in vitro include exposure to Neurotrophin-3 (NT-3) (e.g., at 10 ng/ml) plus All-trans Retinoic Acid (RA) (e.g., at 100 nM), to Glial Growth Factor (GGF) (e.g., at 10 ng/ml), or to Brain Derived Neurotrophic Factor (BDNF) (e.g., at 10 ng/ml). The provided tGRP cells do not produce neurons under these example conditions.

Cell populations were isolated from the dorsal telencephalon based on the antigenic phenotype of restricted precursor cells previously identified in the spinal cord. These telencephalic cells were characterized in mass culture and at the clonal level and were found to generate all macroglial subtypes but were unable to generate neurons under conditions that generally promote neuronal lineage.

The dorsal telencephalon was determined to be capable of generating this glial restricted population de novo by separating the dorsal telencephalon at a time point where the cell populations present are exclusively of a dorsal origin. A ventral glial restricted cell population was detected in parallel.

The ability of the dorsal cell population to differentiate into myelin producing oligodendrocytes upon transplantation in a myelin deficient background was confirmed, as well as GFAP⁺ astrocytes when transplanted into the perinatal forebrain. Thus, described are populations of precursor cells isolated from the embryonic telencephalon that are able to generate both oligodendrocytes and astrocytes but are unable to generate neuronal progeny under conditions that generally promote neuronal lineage.

Also provided is a defined cell population that is generated de novo in the dorsal aspect of the telencephalon and is a source for dorsally derived glial cells. Further provided is a cell population in the telencephalon that can act as a source of astrocytic cells both ventrally as well as dorsally. Thus, disclosed is a model of gliogenesis by which glial cells originate in a timely and organized manner in the developing telencephalon.

Provided herein are compositions and methods for the treatment of CNS injury, including traumatic or degenerative conditions of the CNS, promotion of axon regeneration, suppression of astrogliosis, re-alignment of host tissues, and the delay of axon growth inhibitory proteoglycan expression. Thus, provided are methods of treating a CNS lesion in a subject, comprising administering to the subject a composition comprising telencephalic glial-restricted cell populations and/or cells derived from a telencephalic glial-restricted cell, including tGRP progeny or combinations thereof. tGRP progeny include any GFAP+ cell derived or produced from a tGRP. For example, tGRP progeny include tGRP derived astrocytes, GDAs, and APCs. Optionally, the GDA is a type-1 GDA. Optionally, the astrocyte is a type-1 astrocyte. tGRP progeny also include any GalC+ cell derived or produced from a tGRP. For example, tGRP progeny include oligodendrocytes. Methods of treating a CNS lesion in a subject, comprising administering to the subject an Olig2⁻ cell or cells are also provided. Described cells or combinations thereof can be administered in combination with other compositions as described herein.

The methods can be used for the treatment of spinal cord injury or other CNS injuries. The methods can also be used in CNS lesions in which it is desirable to promote regeneration and/or re-alignment of host tissues, modulate the CNS scarring response, and rescue neurons from atrophy and death, or any combination thereof.

As used herein, the term GDAs (glial restricted precursor derived astrocyte) refers to glial fibrillary acidic protein (GFAP)+/A2B5− cells, also referred to herein as type-1 GDAs, unless type-2 GDAs (GFAP+/A2B5+ cells) are specifically referenced.

The limited success of stem cell and neural precursor cell transplantation is likely due to the inflammatory environment of adult CNS injuries, which direct undifferentiated neural stem cells or glial precursors to a scar astrocyte like phenotype. Scar astrocytes are poorly supportive of axon growth.

Methods and compositions described herein can provide an alternative to allowing the lesion environment to direct differentiation of stem or precursor cells while still retaining the benefit of starting with an undifferentiated cell. Provided herein are methods of treating a CNS lesion in a subject, comprising administering to the subject a composition comprising telencephalic glial restricted precursor cells or cells derived from a tGRP cell. The term lesion is used herein to refer to a site of injury to the CNS, a site of a CNS disease process, degenerative damage, or scarring, wherein promotion of regeneration would provide benefit.

Telencephalic glial-restricted precursor (tGRP) populations can generate oligodendrocytes, APCs, and can preferentially generate type-1 GDAs and type-1 astrocytes versus type 2 astrocytes. tGRP cells are restricted to the glial lineage in vivo as they are unable to generate neuronal phenotypes in an in vivo neurogenic environment. tGRP cells survive and migrate in the neonatal and adult brain. Transplanted tGRP cells can differentiate into myelin-forming oligodendrocytes in a myelin-deficient background and can also generate immature oligodendrocytes in the normal neonatal brain. Transplanted tGRP cells can also differentiate into type-1 GDAs and type-1 astrocytes when administered to a CNS lesion. In some aspects, such transplanted tGRP cells do not produce type-2 astrocytes.

Cell culture technologies can be used for the preparation of tGRPs, APCs, GDAs, astrocytes and oligodendrocytes. As an example, A2B5+ tGRPs can be isolated from dissociated cell suspensions of telencephalon of embryos using standard methods such as, for example, flow cytometry or immunopanning.

tGRPs or tGRP derived APCs, GDAs, astrocytes, or oligodendrocytes can be immortalized by procedures known in the art, so as to preserve a continuing source of tGRPs, or tGRP derived APCs, GDAs, astrocytes, or oligodendrocytes. Immortalized tGRPs or tGRP derived APCs, GDAs, astrocytes, or oligodendrocytes can be maintained in vitro indefinitely. Various methods of immortalization are known in the art including, but not limited to, viral transformation (e.g., with SV40, polyoma, RNA or DNA tumor viruses, Epstein Barr Virus, bovine papilloma virus, or a gene product thereof) and chemical mutagenesis. The cell line can be immortalized by a virus defective in replication, or is immortalized solely by expression of a transforming virus gene product. For example, tGRPs or tGRP derived APCs, GDAs, astrocytes, or oligodendrocytes can be transformed by recombinant expression vectors which provide for the expression of a replication-defective transforming virus or gene product thereof. Such procedures are known in the art.

tGRPs can be maintained in culture in a suitable medium. For example, tGRPs can be maintained in culture with approximately 0.1-100 ng/ml bFGF and SATO supplements on a mixed laminin/fibronectin substrate. In order to differentiate tGRPs to GDAs, the tGRPs can be exposed to, for example, approximately 1-100 ng/ml of recombinant BMP-4 (for approximately 7 days in culture) to differentiate them into GDAs. Also disclosed is the use of other members of the BMP family, or other signaling molecules that induce differentiation along the astrocyte pathway within the antigenic range of type-1 astrocytes.

tGRPs or tGRP derived APCs, GDAs, astrocytes, or oligodendrocytes can be cryopreserved. Various methods for cryopreservation of viable cells are known and can be used (see, e.g., Mazur, 1977, Cyrobiology 14:251-272; Livesey and Linner, 1987, Nature 327:255; Linner, et al., 1986, J. Histochem. Cytochem. 34(9):1123-1135; U.S. Pat. No. 4,199,022 to Senkan et al.; U.S. Pat. No. 3,753,357 to Schwartz; U.S. Pat. No. 4,559,298 to Fahy, which are incorporated by reference at least for the methods described therein).

GDAs for use in the methods described herein can be generated by the method comprising isolating telencephalic cells from the subject, purifying A2B5 positive tGRPs, and culturing said cells with a BMP.

To ensure GDA suspensions for transplantation do not contain undifferentiated tGRPs or cells with the phenotype of type-2 astrocytes, contaminating cell types can be removed from the suspension by, for example, immuno-panning with the A2B5 antibody. A small volume of the resulting suspension can be plated onto glass coverslips and labeled with antibodies to A2B5 and GFAP to verify a uniform type-1 astrocyte phenotype. For transplantation, GFAP positive/A2B5 negative GDAs can be suspended in a suitable medium such as, for example, Hanks Balanced Salt Solution, at a density of 10³-10⁶ cells/μL.

tGRP-derived GDAs can be generated by BMP exposure and fall within the population of cells defined by their antigenic phenotype as type-1 astrocytes. In vitro studies on cells purified from the postnatal CNS have shown that type-1 astrocytes of postnatal origin promote extensive neurite growth from a variety of neurons in vitro, express high levels of axon growth supportive molecules such as laminin/fibronectin and NGF/NT-3 and also exhibit minimal chondroitin sulfate proteoglycan immunoreactivity in vitro. However, while transplantation of immature cortical astrocytes into adult brain injuries or acute adult spinal cord injuries have been shown to suppress astrogliosis, only limited sprouting of endogenous axons have been observed, with axons failing to penetrate the center of grafts or re-enter white matter beyond the sites of injury.

Thus, although GDAs show antigenic phenotypes like type-1 astrocytes, GDAs are a unique cell type that, when transplanted into CNS lesion sites, promote an unprecedented level of tissue reorganization, axon regeneration and locomotor recovery.

GDAs promote robust axon regeneration and functional recovery after transplantation into CNS lesion sites. The ability of GDAs to fill an injury site, suppress astrogliosis, re-align host tissues and delay expression of axon growth inhibitory proteoglycans indicate that these cells possess an effective ability to provide an axon regenerative environment. These attributes, in combination with their striking ability to significantly reduce atrophy of axotomized CNS neurons and support a robust behavioral recovery, make GDAs a highly effective cell type with which to repair a damaged or diseased CNS. Thus, the GDAs can promote axon regeneration, suppress astrogliosis, re-align host tissues, delay expression of axon growth inhibitory proteoglycans, or any combination thereof.

Provided herein is an isolated tGRP cell or a population of isolated tGRP cells. As used herein, the term isolated refers to a cell or population of cells which has been separated from its natural environment, e.g., removal from a donor animal, e.g., human or embryo. The isolated cell or population of cells can be in the form of a tissue sample, e.g., an intact sheet of cells, e.g., a monolayer of cells, or it can be in a cell suspension. The term isolated does not preclude the presence of other cells. The term population is intended to include two or more cells. Cells in a population can be obtained from the same or different source(s).

The telencephalic glial restricted precursor cells can be isolated from a mammal, including an embryo, selected from the group consisting of human and non-human primates, equines, canines, felines, bovines, porcines, ovines, rats and lagomorphs.

Provided herein are isolated cell populations comprising at least about a 10%, 20%, 30%, 40%, 50%. 60%, 70%, 80%, 85%, 90%, 95%, 96%, 97%, 98%, 99%, or 100% pure population of tGRPs or any percent between 10 to 100%. Thus, for example, the isolated cell population can comprise at least 90% tGRPs. The isolated population can also comprise at least 95% tGRPs or at least 99% tGRPs. Cell populations comprising the same percentages of Olig2⁻ GRP cells are also provided. The Olig2⁻ GRP cells are optionally isolated from the dorsal telencephalon.

Optionally, the isolated cell population does not comprise type-2 astrocytes. Optionally, the isolated cell population does not comprise pluripotential or multipotential stem cells, such as ES cells or neuroepithelial stem cells. However, the isolated cell population can also comprise about 0.01%, 0.05%, 0.1%, 0.5%, 1%, 2%, 3%, 4%, 5%, 6%, 7%, 8%, 9%, or 10% type-2 GDAs, type-2 astrocytes, APCs, pluripotential stem cells, multipotential cells, undifferentiated glial precursors, or any combination thereof. Thus, for example, the isolated cell population can comprise less than 10% type-2 GDAs. The isolated cell population can also comprise less than 5% type-2 GDAs. The purity of a cell population can be determined by, for example, detecting markers specific for various cell types in culture and determining by visual observation the percentage of cell types in the population. Also provided are compositions comprising the isolated cell populations in combination with other compositions including compounds, agents or molecules.

A purified population of cells can be grown in feeder-cell-independent culture on a substratum and in a medium configured for supporting adherent growth of the telencephalic glial restricted precursor cells or derivatives thereof and at a temperature and in an atmosphere conducive to growth of the precursor cells and derivatives thereof. The telencephalic glial restricted precursor cells and derivatives can be purified using procedures such as specific antibody capture, fluorescence activated cell sorting, magnetic bead capture, and the like.

Provided herein is an isolated tGRP derivative or progeny cell, or a population of isolated tGRP derivative or progeny cells. Optionally, the tGRP derivative or progeny cell or cells are GFAP+. For example, the derivative or progeny cell or cells can be an APC, type-1 GDA or type-1 astrocyte. In another aspect, the tGRP derivative or progeny cell or cells are GalC+. For example, the tGRP derivative or progeny cell can be an oligodendrocyte.

Thus, provided herein is an isolated APC, GDA, astrocyte or oligodendrocyte cell, or a population of isolated APC, GDA, astrocyte or oligodendrocyte cells, derived from a tGRP, or isolated tGRP population. tGRP derived isolated APC, GDA, astrocyte or oligodendrocyte populations can comprise at least about an 10%, 20%, 30%. 40%, 50%, 60%, 70%, 80%, 85%, 90%, 95%, 96%, 97%, 98%, 99%, or 100% pure population of each respective cell type or any percent between 10% to 100%. Thus, for example, the isolated cell population can comprise at least 90% APCs, GDAs, astrocytes, or oligodendrocytes. The isolated population can also comprise at least 95% APCs, GDAs, astrocytes, or oligodendrocytes or at least 99% APCs, GDAs, astrocytes, or oligodendrocytes. In certain aspects, the isolated cell population does not comprise type-2 astrocytes or type-2 GDAs. Optionally, the isolated cell population does not comprise pluripotential or multipotential stem cells, such as ES cells or neuroepithelial stem cells. However, the isolated cell population of the method can comprise at most about 0.01%, 0.05%, 0.1%, 0.5%, 1%, 2%, 3%, 4%, 5%, 6%, 7%, 8%, 9%, or 10% type-2 GDAs, type-2 astrocytes, pluripotential stem cells, multipotential cells, undifferentiated glial precursors (e.g., GRPs), or any combination thereof. Thus, for example, the isolated cell population can comprise less than 10% type-2 GDAs. The isolated cell population can also comprise less than 5% type-2 GDAs.

The purity of a cell population can be determined by, for example, detecting markers specific for various cell types in culture and determining by visual observation the percentage of cell types in the population. Also provided herein are compositions comprising the isolated cell populations in combination with other compositions including compounds, agents or molecules.

The tGRPs or tGRP derived APCs, GDAs, astrocytes, oligodendrocytes or combinations thereof can be administered using standard methods known in the art for use in the promotion of CNS nerve regeneration and/or scar reduction. The tGRPs or tGRP derived APCs, GDAs, astrocytes, oligodendrocytes or combinations thereof can be administered to treat subjects in which it is desired to promote CNS regeneration and/or reduce scar formation. Thus, tGRPs or tGRP derived APCs, GDAs, astrocytes, oligodendrocytes, or combinations thereof can be applied in any conventional formulation to areas of a lesion.

There is no restriction to the location of a lesion. Thus, any part of the brain or spinal cord can be treated. For example, the cerebral cortex, the mid-brain, the thalamus, the hypothalamus, the striatum, the substantia nigra, the pons, the cerebellum, the medulla, or any cervical, thoracic, lumbar, or sacral spinal segment. The methods are applicable for any nervous system lesion including, for example, those caused by spinal cord injury (resulting, for example, in respiratory paralysis, quadriplegia, and paraplegia).

The tGRPs or tGRP derived APCs, GDAs, astrocytes, oligodendrocytes, or combinations thereof can also be administered to patients in whom the nervous system has been damaged or injured by trauma, surgery, ischemia, infection, metabolic disease, nutritional deficiency, malignancy, toxic agents, paraneoplastic syndromes and degenerative disorders of the nervous system. Examples of such disorders include, but are not limited to, Alzheimer's Disease, Parkinson's Disease, Huntington's chorea, amyotrophic lateral sclerosis, progressive supranuclear palsy, and neuropathies. tGRPs or tGRP derived APCs, GDAs, astrocytes, oligodendrocytes or combinations thereof, can be administered to a wound to reduce scar formation. Thus, after an operation, tGRPs or tGRP derived APCs, GDAs, astrocytes, oligodendrocytes, or combinations thereof, can be administered in order to reduce scar formation from lesions due to, for example, arterio-venous malformation, necrosis, bleeding, and craniotomy, which can secondarily give rise to epilepsy. tGRPs or tGRP derived APCs, GDAs, astrocytes, oligodendrocytes, or combinations thereof, can also be used for treatment of epilepsy, by stabilizing the epileptic focus and reducing scar formation.

Treatment can be performed, for example, within 24 hours, or alternatively, for example, one week, 5 years, or even more than 10 years after onset of the lesion. In cases where a lesion can be predicted, for example, during surgery, the tGRPs or tGRP derived APCs, GDAs, astrocytes, oligodendrocytes, or combinations thereof, can be delivered prior to or during the occurrence.

tGRPs or tGRP derived APCs, GDAs, astrocytes, oligodendrocytes, or combinations thereof, can be delivered by direct application, for example, by direct injection of a sample of tGRPs or tGRP derived APCs, GDAs, astrocytes, oligodendrocytes, or combinations thereof, into the site of neural tissue damage. For example, the spinal cord can be exposed by laminectomy, and a cellular suspension injected using a microsyringe under a surgical microscope. When high resolution MRI images are obtained, the cell suspension can be injected without laminectomy as in intervertebrally (e.g., by the technique of lumbar puncture).

Methods for treating a neurological or neurodegenerative injury comprises administering to a mammal in need of such treatment an effective amount of telencephalic glial restricted precursor cells or derivatives thereof. The tGRP cells or derivatives thereof can be caused to (1) proliferate and differentiate in vitro prior to being administered, or (2) proliferate in vitro prior to being administered and to further proliferate and differentiate in vivo after being administered, or (3) proliferate in vitro prior to being administered and then to differentiate in vivo without further proliferation after being administered, or (4) proliferate and differentiate in vivo after being injected directly after being freshly isolated. The tGRP cells or derivatives thereof can be from a heterologous donor or an autologous donor. The donor can be a fetus, a juvenile, or an adult. The injury to be treated can be multiple sclerosis, spinal cord injury, CNS trauma, conditions in which axonal regeneration is desired, conditions in which control or reduction in glial scarring is desired, any dysmyelinating disorder, or an enzymatic disorder. The tGRP cells, derivatives, or combinations thereof, can be administered locally or widely in the CNS.

Optionally, tGRPs or tGRP derived APCs, GDAs, astrocytes, oligodendrocytes, or combinations thereof, are delivered in a media which partially impedes their mobility so as to localize the tGRPs or tGRP derived APCs, GDAs, astrocytes, oligodendrocytes, or combinations thereof, to a site of lesion. By way of example, tGRPs or tGRP derived APCs, GDAs, astrocytes, oligodendrocytes, or combinations thereof, can be delivered in a paste or gel comprising, for example, a biodegradable gel-like polymer such as fibrin or a hydrogel. Such a semi-solid medium can impede the migration of (scar-producing) undesirable mesenchymal components such as fibroblasts into the site.

Optionally, tGRPs or tGRP derived APCs, GDAs, astrocytes, oligodendrocytes, or combinations thereof, can be administered with the use of polymer implants and surgical bypass techniques. Uses of polymer implants and surgical techniques are known to those of skill in the art. For example, tGRPs or tGRP derived APCs, GDAs, astrocytes, oligodendrocytes, or combinations thereof, can be applied to a site of a lesion in a form in which the tGRPs or tGRP derived APCs, GDAs, astrocytes, oligodendrocytes, or combinations thereof, are seeded or coated onto a polymer implant. Various types of polymer implants can be used herein, with various compositions, pore sizes, and geometries. Such polymers include, but are not limited to, those made of nitrocellulose, polyanhydrides, and acrylic polymers (see e.g., those described in European Patent Publication No. 286284; Aebischer, et al., 1988, Brain Res. 454:179-187; Aebischar, et al., 1988, Prog. Brain Res. 78:599-603; Winn, et al., 1989, Exp. Neurol. 105:244-250, which are incorporated by reference at least for the polymers described therein).

Polymers can be used as synthetic bridges, over which nerve regeneration can be promoted and scar formation can be reduced by application of tGRPs or tGRP derived APCs, GDAs, astrocytes, oligodendrocytes, or combinations thereof, to the end(s), or in the vicinity of, the bridge. For example, an acrylic polymer tube with tGRPs or tGRP derived APCs, GDAs, astrocytes, oligodendrocytes, or combinations thereof, at one or more ends, or throughout the tube, can be used to bridge lesions rostrally or bypass lesions, e.g., of the spinal cord, over which regeneration can be induced. Semi-permeable tubes can be used, e.g., in the dorsal columns or dorsal afferents, which tubes can contain and provide for the release of trophic factors or anti-inflammatory agents. The types of tubes which can be used are well known to those of skill in the art.

Axon fibers that demonstrate regenerative growth or collateral sprouting encounter an inhibitory environment as well as a physical gap that requires a permissive bridging substance. Thus synthetic bridges can be used in the methods described herein. Advances in the field of biomatrix material have provided opportunities to bridge the gap with artificial material, such as biodegradable hydrogels, or combinations of hydrogels and cells, that may promote regeneration. Desired properties of a synthetic bridge are to provide simultaneously a physical substrate for axonal attachment and growth without triggering antigenic host reactions.

Optionally, tGRPs or tGRP derived APCs, GDAs, astrocytes, oligodendrocytes, or combinations thereof, can be administered in combination with other compositions including therapeutic or pharmacological compounds, agents and molecules. For example, several agents have been applied to acute spinal cord injury (SCI) management and CNS lesions that can be used in combination with the compositions and methods. Such agents include agents that reduce edema and/or the inflammatory response. Exemplary agents include, but are not limited to, steroids, such as methylprednisolone; inhibitors of lipid peroxidation, such as tirilazad mesylate (lazaroid); and antioxidants, such as cyclosporin A, EPC-K1, melatonin and high-dose naloxone. Thus, the compositions including tGRPs or tGRP derived APCs, GDAs, astrocytes, oligodendrocytes, or combinations thereof, can further comprise methylprednisolone, tirilazad mesylate, cyclosporin A, EPC-K1, melatonin, or high-dose naloxone or any combination thereof.

The compositions including tGRPs or tGRP derived APCs, GDAs, astrocytes, oligodendrocytes, or combinations thereof, can also comprise, glutamate receptor antagonists including, but not limited to, the noncompetitive N-methyl-D-aspartate (NMDA) ion channel blocker MK-801 (dizocilpine, Merck & Co., Inc., Whitehouse Station, N.J.), 1,2,3,4-tetrahydro-6-nitro-2,3-dioxobenzo[f]quinoxaline-7-sulfonamide (NBQX), gacyclidine (GK-11, Beaufour-Ipsen, Paris, France), and agmatine.

Anti-inflammatory agents, such as, for example, CM101, cytokine IL-10, and selective cyclooxygenase (COX)-2 inhibitors can be used in conjunction with the tGRPs or tGRP derived APCs, GDAs, astrocytes, oligodendrocytes, or combinations thereof. Thus, the compositions can further comprise CM101, IL-10, or a selective COX-2 inhibitor or any combination thereof.

The tGRPs or tGRP derived APCs, GDAs, astrocytes, oligodendrocytes, or combinations thereof, can also be used in conjunction with inhibitors of apoptosis, such as caspase inhibitors, for example, Bcl-2, and calpain inhibitors.

Compositions including tGRPs or tGRP derived APCs, GDAs, astrocytes, oligodendrocytes, or combinations thereof, can also comprise exogenous neurotrophins, including, but not limited to, nerve growth factor (NGF), glial-derived neurotrophic factor (GDNF), cilliary neurotrophic factor (CNTF), neurotrophic factor-3 and 4/5 (NT-3, NT-4/5), fibroblastic growth factor (FGF), and brain-derived neurotrophic factor (BDNF) or any combination thereof.

Inhibitors of netrins, semaphorins, ephrins, tenascins, integrins, and chondroitin sulfate proteoglycans (CSPG) can be used in combination with tGRPs or tGRP derived APCs, GDAs, astrocytes, oligodendrocytes, or combinations thereof. For example, chondroitinase can be used to remove CSPG. Thus, the compositions can further comprise an inhibitor of netrins, semaphorins, ephrins, tenascins, integrins, or CSPG. Thus, the compositions can further comprise a chondroitinase.

The compositions including tGRPs or tGRP derived APCs, GDAs, astrocytes, oligodendrocytes, or combinations thereof, can also comprise, the IN-1 antibody, which neutralizes the inhibitory protein activity of NoGo, the myelin-derived growth-inhibitory protein, myelin-associated glycoprotein (MAG) or any combination thereof.

Agents that act through direct intracellular mechanisms in the nerve cell body to promote neurite growth can be used in combination with tGRPs or tGRP derived APCs, GDAs, astrocytes, oligodendrocytes, or combinations thereof. Thus, inosine, a purine nucleoside, and cAMP and the compound AIT-082, a synthetic hypoxanthine derivative containing a para-aminobenzoic acid moiety (e.g., Neotrofin; NeoTherapeutics, Newport Beach, Calif.) can be used in the compositions and methods. Thus, the compositions can further comprise AIT-082.

Gene therapy allows the engineering of cells, which combines the therapeutic advantage of the cells in combination with a gene delivery system. For example, if delivery of neurotrophins is desired, cells that form myelin and secrete neurotrophins can be engineered to both promote neurite growth and restore nerve function.

Macrophages from the patient's own blood (autologous macrophages) can be activated and implanted at the site of the injury in combination with tGRPs or tGRP derived APCs, GDAs, astrocytes, oligodendrocytes, or combinations thereof. The patient's own activated macrophages can scavenge degenerating myelin debris, rich in non-permissive factors, and thus encourage regenerative growth without eliciting an immune response.

The compositions including tGRPs or tGRP derived APCs, GDAs, astrocytes, oligodendrocytes, or combinations thereof, can further comprise immuno-suppressive drugs such as cyclosporins, tacrolimus (FK505), cyclophosamid, azathioprines, methotrexate, mizoribin alone or in any combination or the use thereof. Thus, the compositions can further comprise cyclosporins, tacrolimus (FK505), cyclophosamid, azathioprines, methotrexate, or mizoribin.

Administration of any composition in combination with the administration of tGRPs or tGRP derived APCs, GDAs, astrocytes, oligodendrocytes, or combinations thereof, can be performed prior to, concurrent with, or after the administration of a tGRPs or tGRP derived APCs, GDAs, astrocytes, oligodendrocytes or a combination thereof. Thus, the methods described herein can further comprise, administration of a composition including agents, compounds or molecules, prior to, during, or after administration of the tGRPs or tGRP derived APCs, GDAs, astrocytes, oligodendrocytes, or combinations thereof. The compositions and methods described herein may comprise a composition including agents, compounds or molecules in any combination. By way of example, the compositions containing tGRPs or tGRP derived APCs, GDAs, astrocytes, oligodendrocytes, or combinations thereof, described herein may also comprise a glutamate receptor antagonist and a neurotrophin. One or more of the compositions including agents, compounds or molecules can be formulated with the tGRPs or tGRP derived APCs, GDAs, astrocytes, oligodendrocytes, or combinations thereof, containing composition or can be administered separately from the tGRPs or tGRP derived APCs, GDAs, astrocytes, oligodendrocytes, or combinations thereof, containing compositions described herein. If administered separately, the one or more additional composition including agents, compounds or molecules can be administered before, after or simultaneously with the tGRPs or tGRP derived APCs, GDAs, astrocytes, oligodendrocytes, or combinations thereof, containing compositions as appropriate.

Any combination of composition including agents, compounds or molecules, or therapies can be combined with the tGRPs or tGRP derived APCs, GDAs, astrocytes, oligodendrocytes, or combinations thereof, described herein even if not explicitly mentioned as a combination. For example, combinations of immunosuppressive drugs and tGRPs or tGRP derived APCs, GDAs, astrocytes, oligodendrocytes, or combinations thereof, can further include any other agent mentioned herein (e.g., bridges, neurotrophic factors and/or anti-inflammatory agents).

The number of tGRPs or tGRP derived APCs, GDAs, astrocytes, oligodendrocytes, or combinations thereof, to be administered can depend on the species, age, weight and the extent of the lesion(s). Optionally, administered doses range from about 10³-10⁸, including 10³-10⁵, 10⁵-10⁸, 10⁴-10⁷, cells or any amount in between in total for an adult patient.

An effective amount of tGRP cells or derivatives thereof or mixtures thereof for administration refers to an amount or number of cells sufficient to obtain the selected effect. For example, an effective amount of tGRP cells for treating scarring can be an amount of cells sufficient to obtain a measurable decrease in the amount of scarring. tGRP cells can generally be administered at concentrations of about 5-50,000 cells/microliter. Optionally, administration can occur in volumes up to about 15 microliters per injection site. However, administration to the central nervous system can involve volumes many times this size.

As used herein treating or treatment does not have to mean a complete cure. It can also mean that one or more symptoms of the underlying disease are reduced, and/or that one or more of the underlying cellular, physiological, or biochemical causes or mechanisms causing the symptoms are reduced. It is understood that reduced, as used in this context, means relative to the state of the disease, including the molecular state of the disease, not just the physiological state of the disease.

When the terms prevent, preventing, and prevention are used herein in connection with a given treatment for a given condition (e.g., prevention of a CNS lesion), they mean that the treated subject either does not develop an observable level of the condition at all, or develops it more slowly and/or to a lesser degree than he/she would have absent the treatment. These terms are not limited solely to a situation in which the subject experiences no aspect of the condition whatsoever. For example, a treatment can be said to have prevented the condition if it is given during exposure of a subject to a stimulus that would have been expected to produce a given manifestation of the condition, and results in the subject's experiencing fewer and/or milder symptoms of the condition than otherwise expected. A treatment can prevent lesions of the CNS, for example, by resulting in the subject's displaying only mild overt symptoms of the lesion.

The compositions including agents, compounds or molecules can be delivered at effective amounts or concentrations. An effective concentration or amount of a substance is one that results in treatment or prevention of lesions of the CNS, promotion of axon regeneration, suppression of astrogliosis, re-alignment of host tissues, and the delay of axon growth inhibitory proteoglycans expression. The term therapeutically effective means that the amount of the composition used is of sufficient quantity to ameliorate one or more causes or symptoms of a disease or disorder. Such amelioration only requires a reduction or alteration, not necessarily elimination.

Effective dosages and schedules for administering the compositions can be determined empirically. The dosage ranges for the administration of the compositions are those large enough to produce the desired effect in which the symptoms disorder are affected. The dosage should not be so large as to cause adverse side effects, such as unwanted cross-reactions, anaphylactic reactions, and the like. The exact amount of the compositions required can vary from subject to subject. Generally, the dosage can vary with the age, condition, sex and extent of the disease in the patient, route of administration, or whether other drugs are included in the regimen, and can be determined by one of skill in the art. The dosage can be adjusted by the individual physician in the event of any counter indications. Dosage can vary, and can be administered in one or more dose administrations daily, for one or several days. Guidance can be found in the literature for appropriate dosages for given classes of pharmaceutical products.

The provided tGRPs or tGRP derived APCs, GDAs, astrocytes, oligodendrocytes, or combinations thereof, can be prepared by making cell suspensions of the cultured tGRPs or tGRP derived APCs, GDAs, astrocytes, or oligodendrocytes in a culture medium or a pharmaceutically acceptable carrier. Cell density for application can be from about 10³-10⁶ cells/μL. Thus, provided herein is a pharmaceutical composition comprising an effective amount of the disclosed tGRPs or tGRP derived APCs, GDAs, astrocytes, oligodendrocytes, or combinations thereof, in a pharmaceutically acceptable carrier.

The term carrier means a compound, composition, substance, or structure that, when in combination with a compound or composition, aids or facilitates preparation, storage, administration, delivery, effectiveness, selectivity, or any other feature of the compound or composition for its intended use or purpose. For example, a carrier can be selected to minimize any degradation of the active ingredient and to minimize any adverse side effects in the subject. Such pharmaceutically acceptable carriers include sterile biocompatible pharmaceutical carriers, including, but not limited to, saline, buffered saline, dextrose, and water.

The compositions for use with the tGRPs or tGRP derived APCs, GDAs, astrocytes, or oligodendrocytes or combinations thereof, including agents, compounds or molecules can be incorporated into microparticles, liposomes, or cells. Any of the microparticles, liposomes or cells, including the tGRPs or tGRP derived APCs, GDAs, astrocytes, oligodendrocytes, or combinations thereof, can be targeted to a particular cell type via antibodies, receptors, or receptor ligands. Targeting can be accomplished by various means known to those of skill in the art, including, for example, by way of genetic engineering.

Suitable carriers and their formulations are described in Remington: The Science and Practice of Pharmacy (21th ed.) Lippincott Williams & Wilkins (2005). Examples of the pharmaceutically-acceptable carrier include, but are not limited to, saline, Ringer's solution and dextrose solution. The pH of the solution can be from about 5 to about 8 or from about 7 to about 7.5. Further carriers include sustained release preparations such as semi-permeable matrices of solid hydrophobic polymers, which matrices are in the form of shaped articles, e.g., films, liposomes or microparticles. Preparations for parenteral administration include sterile aqueous or non-aqueous solutions, suspensions, and emulsions. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers and electrolyte replenishers (such as those based on Ringer's dextrose). Preservatives and other additives can also be present such as, for example, antimicrobials, anti-oxidants, chelating agents, and inert gases.

Delivery systems for other optional compositions, such as neurotrophic factors, include administration by direct injections through catheters attached to indwelling osmotic pumps, through genetically engineered biological delivery systems such as transduced fibroblasts or immortalized cell lines, and by direct injection of genes or proteins into the spinal parenchyma at or near the lesion site.

Parenteral administration of the compositions can be accomplished by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

Disclosed herein are kits that are drawn to reagents that can be used in practicing the methods disclosed herein. The kits can include any reagent or combination of reagents that would be understood to be required or beneficial in the practice of the disclosed methods. For example, the kits could include tGRPs or tGRP derived APCs, GDAs, astrocytes, oligodendrocytes, or combinations thereof, as well as, buffers and compositions for using them. Other examples of kits, include tGRPs or tGRP derived APCs, GDAs, astrocytes, oligodendrocytes, or combinations thereof, described herein, as well as neurotrophic factors, such as NGF, as well as the buffers and compositions for using them. Optionally, kits include tGRPs or tGRP derived APCs, GDAs, astrocytes, oligodendrocytes, or combinations thereof, and instructions to use the same in the methods described herein.

The disclosed methods and compositions are applicable to numerous areas including, but not limited to, the treatment of CNS lesions. The disclosed compositions and methods can also be used in a variety of ways as research tools. Other uses are disclosed, apparent from the disclosure, and/or will be understood by those in the art.

Disclosed are materials, compositions, and components that can be used for, can be used in conjunction with, can be used in preparation for, or are products of the disclosed methods and compositions. These and other materials are disclosed herein, and it is understood that when combinations, subsets, interactions and groups of these materials are disclosed that while specific reference of each various individual and collective combinations and permutation of these compounds may not be explicitly disclosed, each is specifically contemplated and described herein. For example, if a cell is disclosed and discussed and a number of modifications that can be made including the cell are discussed, each and every combination and permutation of the cell and the modifications that are possible are specifically contemplated unless specifically indicated to the contrary. Thus, if a cell type A, B, and C are disclosed as well as a cell type D, E, and F and an example of a combination of cells, A-D is disclosed, then even if each is not individually recited, each is individually and collectively contemplated. Thus, this example, each of the combinations A-E, A-F, B-D, B-E, B-F, C-D, C-E, and C—F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this application including, but not limited to, steps in methods of making and using the disclosed compositions. Thus, if there are a variety of additional steps that can be performed it is understood that each of these additional steps can be performed with any specific element or combination of elements of the disclosed methods, and that each such combination is specifically contemplated and should be considered disclosed.

Ranges can be expressed herein as from about one particular value, and/or to about another particular value. When such a range is expressed, this includes a range from the one particular value and/or to the other particular value. It will be further understood that the endpoints of each of the ranges are significant both in relation to the other endpoint, and independently of the other endpoint. It is also understood that there are a number of values disclosed herein, and that each value is also herein disclosed as about that particular value in addition to the value itself. For example, if the value 10 is disclosed, then about 10 is also disclosed. It is also understood that each unit between two particular units are also disclosed. For example, if 10 and 15 are disclosed, then 11, 12, 13, and 14 are also disclosed.

As used throughout by a subject is meant an individual. Thus, the subject can include, for example, domesticated animals, such as cats and dogs, livestock (e.g., cattle, horses, pigs, sheep, and goats), laboratory animals (e.g., mice, rabbits, rats, and guinea pigs) mammals, non-human mammals, primates, non-human primates, rodents, birds, reptiles, amphibians, fish, and any other animal. The subject can be a mammal such as a primate or a human.

Optional or optionally means that the subsequently described event or circumstance may or may not occur, and that the description includes instances where said event or circumstance occurs and instances where it does not.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

Unless defined otherwise, all technical and scientific terms used herein have the same meanings as commonly understood by one of skill in the art to which the disclosed method and compositions belong. No admission is made that any reference constitutes prior art. The discussion of references states what their authors assert, and applicants reserve the right to challenge the accuracy and pertinency of the cited documents. It will be clearly understood that, although a number of publications are referred to herein, such reference does not constitute an admission that any of these documents forms part of the common general knowledge in the art.

### EXAMPLES

### Example 1

### Materials and Methods

Cell culture. A2B5+/PSA-NCAM− cells were isolated from embryonic day 15 (E15) Sprague Dawley rat telencephala using A2B5 and an antibody recognizing the polysialylated form of neural cell adhesion molecule (PSA-NCAM) (Rao et al., PNAS 95:3996-4001 (1998); Rao and Mayer-Proschel, Dev. Biol. 188:48-63 (1997); and Mayer-Proschel et al., Neuron 19:773-785 (1997)) in combination with magnetic separation using Miltenyi MACS Cell Separation Columns (Miltenyi Biotech, Auburn, Calif.). For explant studies, the dorsal telencephala was removed from E13 Sprague Dawley rats and placed on Millicell culture plate inserts for two days of in vitro growth in GIBCO® Neural Basal Media (Invitrogen, Carlsbad, Calif.) with the addition of 2 mM GIBCO® Glutamax (Invitrogen, Carlsbad, Calif.) and GIBCO® B27 Supplement minus AO (Invitrogen, Carlsbad, Calif.), before being immunopurified as above. Cells were grown on fibronectin/laminin-coated glass coverslips at 1000 cells per well of a 24 well plate for mass culture experiments or at 500 cells per T25 flask and/or 40 cells per well of a 24 well plate for clonal analysis. For propagation, cultures were grown in DMEM-F12 supplemented with additives as described (Bottenstein and Sato, PNAS 76:514-7 (1979)) and basic fibroblast growth factor (bFGF: 10 ng/ml). At the specified time, cells were stained with A2B5 antibody (Schnitzer and Schachner, Cell Tissue Res. 224:625-36 (1982)) to detect precursor cells, anti-galactocerebroside (GalC) (Bansal et al., J. Neurosci. Res. 24:548-57 (1989)) to identify oligodendrocytes, anti-GFAP antiserum to identify astrocytes (Bignami and Dahl, Brain Res. 49:393-402 (1973) and Norton and Farooq, Brain Res. Dev. Brain Res. 72:193-202 (1993)) and anti-beta III tubulin (Caccamo et al., Lab Invest. 60:390-8 (1989)) to detect neurons, followed by the appropriate fluorochrome conjugated secondary antibodies (Molecular Probes, Inc., Eugene, Oreg.).

Mass culture and clonal analysis of telencephalon populations. Mass culture and clonal differentiation analyses were used to confirm the differentiation potential of cell populations and individual precursor cells, respectively, as used previously in GRP cell characterization from the spinal cord (Rao et al., PNAS 95:3996-4001 (1998); Herrera et al., Exp. Neurol. 171:11-21 (2001); and Mayer-Proschel et al., Neuron 19:773-785 (1997)), as well as in characterization of OPCs (Ibarrola and Rodriguez-Pena, Bran Res. 752:285-293 (1997) and Smith et al., PNAS 97:10032-7 (2000)). Cells were isolated as described above and grown in bFGF for 1 week prior to replating for mass culture or clonal density. Cells were propagated in bFGF for 2 days prior to exposure to one of the following conditions: 10 ng/ml bFGF (control: proliferative), 10 ng/ml Bone Morphogenic Protein 4 (BMP-4: astrocyte induction), 1% Fetal Bovine Serum (FBS: astrocyte induction), 1 ng/ml Platelet Derived Growth Factor (PDGF-AA) plus a mixture of 49 nM Triiodothyronine and 45 nM Thyroxine (PDGF-AA+T3/T4: oligodendrocyte induction), or 10 ng/ml Neurotrophin-3 plus 100 nM Retinoic Acid (NT3+RA: neuron induction).

Section preparation. Embryos from various developmental ages were immersed in cold isopentane (Sigma-Aldrich, St. Louis, Mo.) and stored at −80° C. until sectioned. 10 μm sections were cut using a Shandon Cryotome Cryostat and collected on Superfrost Plus slides (VWR, West Chester, Pa.). Slides were air dried at room temperature overnight and processed for primary antibody staining or stored at −80° C. Sections were fixed by immersion in 4% paraformaldehyde for 10 minutes at room temperature followed by a 2 minute acetone exposure at −20° C. All washing steps were carried out in Tris buffered saline. Blocking buffer consisted of 0.5M TBS with 5% Goat Serum and 4% Bovine Serum Albumin.

Fluorescence Activated Cell Sorting Analysis. Freshly dissociated cells were stained with primary antibodies that included anti-PSA-NCAM with a secondary anti-IgM-PE conjugate, and A2B5 conjugated directly to fluorescein. FACS staining was conducted at 4° C. in the following sequence: Primary PSA-NCAM, secondary IgM-PE, primary A2B5-FITC. Flow cytometry was performed on a Becton Dickinson FACSCalibur™ (Becton Dickinson, Franklin Lakes, N.J.) and analysis was done using CELLQuest™ software (Becton Dickinson, Franklin Lakes, N.J.).

Immunostaining of cells and sections. All primary antibody stains were done at 4° C. overnight, followed by a 30 minute stain with the appropriate secondary. A2B5, PSA-NCAM, 04, Ran2 and GalC hybridoma supernatants (American Type Culture Collection, Manassas, Va.) were used at 1:10 dilutions. 3CB2 and RC2 hybridoma supernatants (Developmental Studies Hybridoma Bank, Iowa City, Iowa) were used at 1:50. GFAP rabbit polyclonal antibody (Dako, Denmark) and beta III tubulin (BioGenex, San Ramon, Calif.) were used at 1:400. Sox2 (Millipore, Temecula, Calif.), Sox10 (Sigma-Aldrich, St. Louis, Mo.), Nestin (Rat 401; Millipore, Temecula, Calif.), NG2 (Millipore, Temecula, Calif.) and PDGFR alpha (Santa Cruz Biotechnology, Santa Cruz, Calif.) antibodies were used at 1:500. CD44 antibody (Accurate Chemical, Westbury, N.Y.) and human Placental Alkaline Phosphatase antibody (Sigma-Aldrich, St. Louis, Mo.) were used at 1:1000. Olig2 antibody (Takebayashi et al., Mechanisms of Development 99:143-8 (2000)) was used at 1:40,000. All secondary antibodies were purchased from Molecular Probes and included goat anti-mouse IgG3, IgM, IgG2a, and goat anti-rabbit Ig (heavy and light chain) conjugated to Alexa-488, Alexa-350, Alexa-546 or Alexa-568.

Clonal splitting experiments. Immunopurified cells were plated at clonal density and grown in 10 ng/ml bFGF until clones were detected containing approximately 200 cells. These clones were then selectively passaged and split into four separate wells containing one of the following: 10 ng/ml bFGF, 1% FBS, 1 ng/ml PDGF-AA plus a mix of 45 nM T3 and 49 nM T4, or 10 ng/ml NT-3 plus 100 nM RA. Media was changed every other day for six days and cells were processed for immunostaining as indicated above.

Transplantation. Postnatal day 18 homozygous shiverer mice were anesthetized with 25 μl of a 100 μg/μl solution of ketamine prior to transplantation. A 0.34 mm needle was used to inject 1.5 μl of PBS containing 1×10⁵ A2B5+/PSA-NCAM− cells at four injection sites lateral to the cortical hem of the left hemisphere. The needle was inserted to a depth of 3 mm and remained in the injection site for 1 minute prior to removal. Shiverer mice undergoing the transplantation procedure were sacrificed three weeks post-transplantation for analysis. Postnatal day 0 Sprague Dawley rat pups were anesthetized by hypothermia for hPAP expressing, telencephalic cell transplantation. 8-9 sites were injected with 27.6 nl per injection site at a depth of 1 mm into the left hemisphere. Rat pups receiving cell transplantations were sacrificed at postnatal day 10 and processed for immunofluorescence as described above.

Electron Microscopy. Animals that underwent cell transplantation were perfused with a mixture of paraformaldehyde and gluteraldehyde warmed to 38° C. Brains were removed and sectioned into 1 mm coronal sections using a Braintree Scientific (Braintree, Mass.) 1 mm mouse acrylic matrix. Each section was fixed overnight in paraformaldehyde/gluteraldehyde mix, rinsed with phosphate buffer, pH 7.4, and post-fixed in phosphate buffered 1.0% osmium tetroxide for 1.5 hours. The 1 mm sections were dehydrated in a graded series of ethanol (ETOH) to 100%, transitioned into 100% propylene oxide and infiltrated in Epon/Araldite (Electron Microscopy Sciences, Fort Washington, Pa.) epoxy resin overnight. Sections were embedded into molds with fresh resin and polymerized for two days at 70° C. Semi-thin two micron sections were cut and stained with 0.5% toluidine blue in 1% sodium borate and examined under a light microscope to determine the area to be thin sectioned. Thin sections were cut with a diamond knife and placed on 200 mesh copper grids and stained with uranyl acetate and lead citrate. The grids were examined with a Hitachi 7100 Transmission Electron Microscope (Tokyo, Japan) and digital images were captured using a MegaView III digital camera (AnalySIS, Lakewood, Colo.).

Results

A2B5⁺ Cells can be Detected in the Dorsal Telencephalon Outside of the Ventral Olig2 Domain.

The dorsal telencephalon was used to pursue initial identification of a glial restricted precursor in the telencephalon as this region provides two major advantages over the ventral telencephalon for cell identification: First, OPCs are not detected in the dorsal telencephalon until after E15 (based on PDGFR-alpha expression), while the ventral telencephalon has been reported to contain OPCs (defined as PDGFR-alpha⁺ cells) as early as E12.5. As both GRPs and OPCs are A2B5⁺, an initial distinction between these two cell types necessitated cell isolation from a specific developmental window in a region such as the E15 dorsal telencephalon, known to possess gliogenic potential but being devoid of the OPC. Second, the dorsal telencephalon consists entirely of dorsal born cells until the time of ventral cell infiltration, at approximately E13.5 in the rat, providing the opportunity to explore the origin of an identified precursor population.

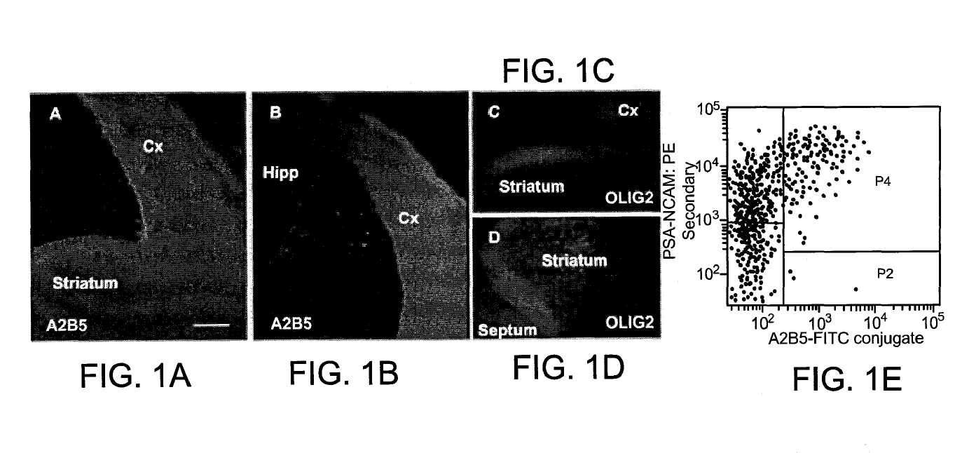

First characterized was the distribution of A2B5⁺ cells in the embryonic telencephalon, and as shown in FIGS. 1A and B, A2B5 labeled cells are present in both the E15 dorsal and ventral telencephalon, whereas Olig2, a marker for OPCs, was found only in the ventral telencephalon (FIGS. 1C and D). To determine the presence of a glial restricted precursor population among the widely A2B5 positive telencephalon, cell isolation and sorting was conducted using the antigenic phenotype that defines spinal cord GRP cells: A2B5⁺/PSA-NCAM⁻. As A2B5 and anti-PSA-NCAM are both IgM antibodies, the A2B5 primary antibody directly conjugated to fluorescein was used allowing for simultaneous labeling of A2B5 and anti-PSA-NCAM immunoreactive cells. FACS analysis revealed three distinct cell populations: PSA-NCAM⁺ only cells, A2B5⁺ only cells, and cells that co-label with anti-PSA-NCAM and A2B5 (FIG. 1E). These results confirm the presence of an A2B5⁺/PSA-NCAM⁻ cell population in the dorsal telencephalon located outside of the Olig2 domain. The A2B5⁺ only population was the focus of further analysis as this antigenic phenotype is shared by the previously identified spinal cord GRP cell. It is important to note, however, that both the A2B5⁺/PSA-NCAM⁺ and the PSA-NCAM⁺ only populations contained at least a subset of cells capable of glial cell generation, as seen in preliminary mass culture experiments.

A2B5 Labels a Subset of Neurons in the Dorsal Telencephalon

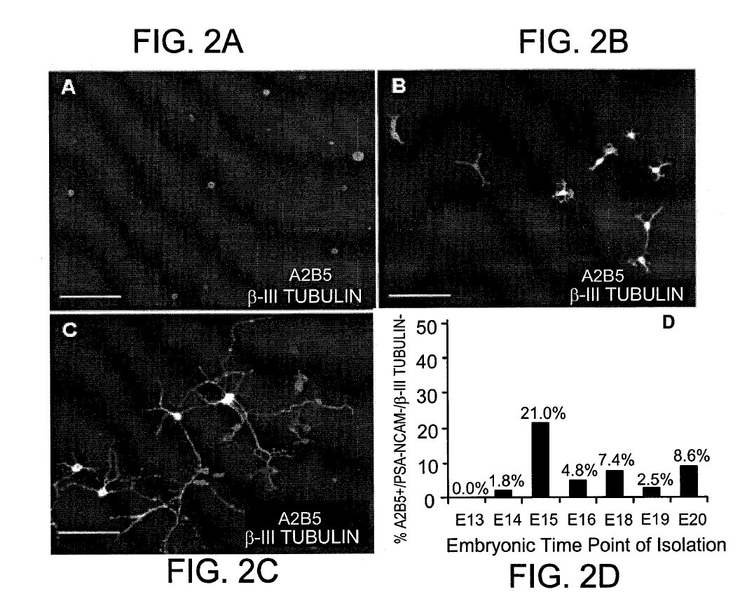

The purification of A2B5⁺/PSA-NCAM⁻ cells from the E15 dorsal telencephalon yielded a heterogeneous population of putative glial precursors and neurons. A2B5⁺/PSA-NCAM⁻ populations isolated as early as E13 to as late as E20 from the dorsal telencephalon contained A2B5⁺ cells expressing the neuronal marker beta III tubulin, detected by immunofluorescence at 4 hours, 12 hours and 4 days post-dissection (FIG. 2A-C). The lack of glial precursor-restricted labeling with A2B5 prompted examination of the A2B5⁺/PSA-NCAM⁻ cell populations in combination with beta III tubulin to determine the appropriate developmental time point that would yield specifically A2B5⁺/PSA-NCAM⁻/beta III tubulin⁻ cells. Acute staining of cells directly after dissection indicated that the peak time for isolating an optimal number of A2B5⁺/PSA-NCAM⁻/beta III tubulin⁻ cells was E15, when A2B5⁺/beta III tubulin⁻ cells represented approximately 21% of the subpopulation of A2B5⁺/PSA-NCAM⁻ E15 dorsal telencephalic cells (FIG. 2D). This time point as the peak time to isolate a putative glial restricted precursor population identified as A2B5⁺/PSA-NCAM⁻/beta III tubulin⁻.

Defining the A2B5⁺/PSA-NCAM⁻/Beta III Tubulin⁻ Population

To further characterize the antigenic profile of the A2B5⁺/PSA-NCAM⁻/beta III tubulin⁻ putative glial restricted precursor population, freshly isolated and MACS sorted cells were allowed to adhere to a FN/LN coated surface over a maximum of 8 hours. Cells were then stained with antibodies directed against spatially relevant and cell-type specific antigens. Table 1 provides a summary of the antibodies used and the determined presence or absence of their respective antigens in the putative glial restricted precursor population.

TABLE 1
Antigenic profile of the A2B5+/PSA-NCAM− population,
pre- and post-in vitro growth

| | | In vitro expanded |
|---|---|---|
| | Freshly isolated | A2B5+/ |
| Antigen | A2B5+/PSA-NCAM− cells | PSA-NCAM− cells |
| A2B5 | + | + |
| CD44 | − | − |
| GFAP | − | − |
| Nestin | + | + |
| NG2 | − | − |
| O4 | − | − |
| Olig2 | − | − |
| PDGFR alpha | − | − |
| PSA-NCAM | − | − |
| Ran2 | − | − |
| S100 | − | − |
| 3CB2 | − | − |
| Sox2 | + | + |
| Sox10 | − | − |
| Beta III Tubulin | + | − |

More mature glial markers were absent as expected, including Olig2, PDGFR alpha, NG2, GFAP, CD44 and SOX10, Ran2 and O4. Antigens associated with neurons and their precursors including NeuN and Doublecortin were not detected. Cells were also negative for the radial glial markers RCB2 and RC2. In contrast to the absence of neuronal markers and more mature glial lineage markers, putative glial restricted precursor population were immunoreactive for both Nestin and Sox2, antigens that have been shown to be present in various populations of stem cells and in GRP cells. While the antigenic profile of the A2B5⁺/PSA-NCAM⁻/beta III tubulin⁻ cell population was not consistent with OPCs, the expression of Nestin and Sox2 did not allow for distinguishing between stem cells and GRP cells. As stem cells differ from GRP cells in their differentiation potential in vitro and in vivo, a number of experiments were conducted that were geared towards the identification of the differentiation potential of the A2B5⁺/PSA-NCAM⁻/beta III tubulin⁻ cell pool. To determine a possible lineage restriction of the A2B5⁺/PSA-NCAM− cell population, the defined cell pool was calculated over a minimum of 7 days in a defined condition that allowed the expansion of the cells without changing their phenotype.

To establish such a condition, freshly isolated, MACS sorted A2B5⁺/PSA-NCAM⁻ cells (comprised of a heterogeneous population of A2B5⁺/PSA-NCAM⁻/beta III tubulin⁺ and of A2B5⁺/PSA-NCAM⁻/beta III tubulin⁻) were plated in defined medium supplement with bFGF and cultured for 7 days. During this culture period, the cells were passaged twice, which resulted in a loss of the A2B5⁺/PSA-NCAM⁻/beta III tubulin⁺ neuronal population. The loss of this neuronal population was attributable to two factors: (i) the medium condition was not permissive for the survival of the neuronal A2B5⁺/PSA-NCAM⁻/beta III tubulin⁺ cells, but was sufficient to allow survival and proliferation of the non-neuronal A2B5⁺/PSA-NCAM⁻/beta III tubulin⁻ population, and (ii) a difference in substrate binding between the neuronal and putative glial precursor populations. To confirm that the loss of the neuronal population was due to cell death, the neuronal A2B5⁺/PSA-NCAM⁻/beta III tubulin⁺ cells were cultured in the presence of PDGF-AA, a factor that has been shown to support neuronal survival. This condition was supportive of the survival of A2B5⁺/PSA-NCAM⁻/beta III tubulin⁺ neurons (as determine by immunofluorescence) but did not support the survival of the non-neuronal A2B5⁺/PSA-NCAM⁻/beta III tubulin⁻ cell pool. The observed difference in substrate binding of the neuronal A2B5⁺/PSA-NCAM⁻/beta III tubulin cells compared to the non-neuronal A2B5⁺/PSA-NCAM⁻/beta III tubulin⁻ cells resulted in the neuronal cells tightly binding to the growth substrate while the non-neuronal cells could be removed with ease, allowing for selective passaging. As a direct application of the above findings, growth of the freshly isolated A2B5⁺/PSA-NCAM− population (containing both putative glial restricted precursors and neurons) in 10 ng/ml bFGF alone resulted in preferential survival of the non-neuronal A2B5⁺/PSA-NCAM⁻/beta III tubulin⁻ population.

To determine whether the cells remained unchanged during in vitro growth, the resultant population that was grown for 7 days as describe above and passaged twice were stained with the antibodies listed in Table 1 and compared to freshly isolated cells. The antigenic profile of the cell population that underwent growth and expansion in bFGF in vitro was identical to the antigenic profile of freshly isolated and MACS sorted cells (see Table 1). Importantly, the A2B5⁺/PSA-NCAM⁻/beta III tubulin⁻ cell population remained Olig2 negative (even after 3 weeks of in vitro growth in basal media supplemented with 10 ng/ml bFGF). This observation is important as it has been suggested by Gabay et al that bFGF might have a “ventralizing” effect on Olig2 negative dorsal derived spinal cord cells. Results did not suggest such a role of bFGF in the dorsal-derived telencephalic A2B5⁺/PSA-NCAM⁻/beta III tubulin⁻ cells. In addition, no spontaneously appearing beta III tubulin⁺ cells or any obvious differences in cell morphology, growth rate, or survival during this in vitro growth, further arguing against the “ventralizing” effects in response to bFGF as described by Gabay et al., 2003.

The A2B5⁺/PSA-NCAM⁻ Population Generates Astrocytes and Oligodendrocytes in Mass Culture but does not Generate Neurons

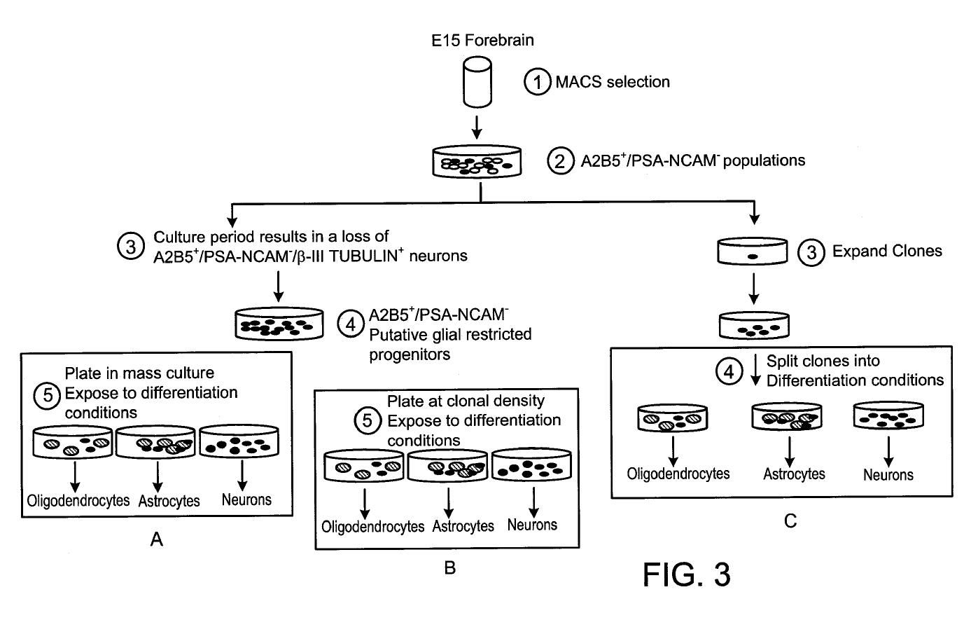

The culture conditions identified allowed for the expansion of cells while maintaining their antigenic phenotype. This in vitro culture system was used to determine whether the A2B5⁺/PSA-NCAM⁻/beta III tubulin⁻ population represented neural stem cells or lineage restricted precursor cells. While both cells population share a similar antigenic profile, their in vitro and in vivo differentiation potential were fundamentally different. Neural stem cells are considered to be multipotent and are able to give rise to glial as well as neuronal populations. In contrast, lineage restricted cells have lost their multipotency and are restricted in their differentiation potential to either glial or neuronal lineages or to a specific subset of cells of either lineage. To determine the differentiation potential of the A2B5⁺/PSA-NCAM⁻/beta III tubulin⁻ cell population from the E15 dorsal telencephalon, mass culture analyses (as shown in FIG. 3A), clonal analyses (3B), and clonal splitting analyses (3C) were conducted. Each experiment was designed to determine the ability of the isolated cell populations to generate astrocytes, oligodendrocytes and neurons. Differentiation conditions used for these analyses were based on our previous data on spinal cord derived GRPs and on many reposts in the literature. As a pro-astrocyte condition, cells were exposed to 1% FBS. To determine whether cells are capable of generating oligodendrocytes, cultures were exposed to PDGF-AA plus T3/T4 (pro-oligodendrocye). To facilitate neuronal differentiation cells, were exposed to NT3 plus RA (pro-neuron), a condition that has been shown to be effective in directing beta III tubulin⁺ neuron formation from spinal cord NEP cells. Control cultures were kept in bFGF and represented the proliferate condition.

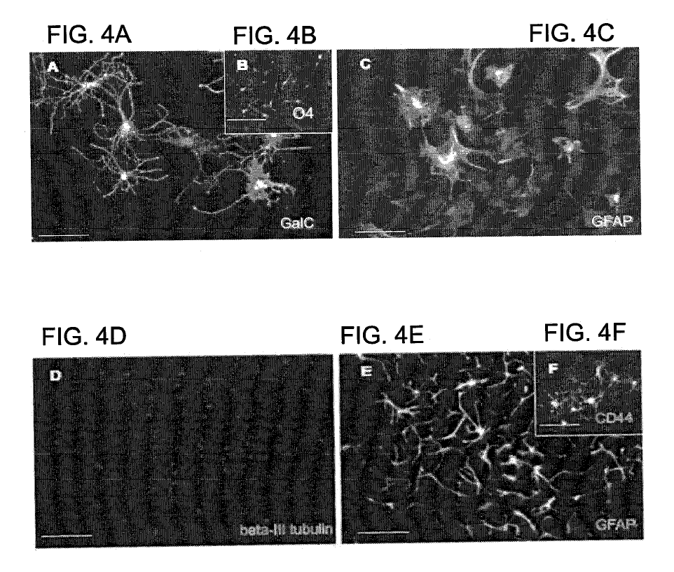

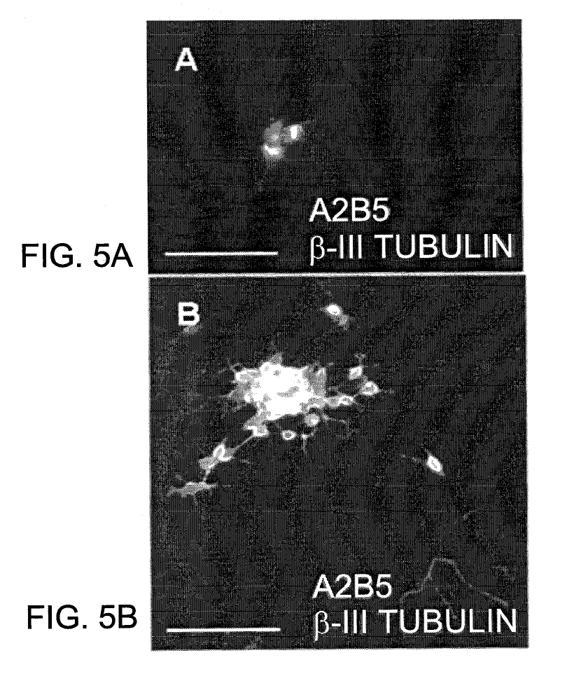

Cells were isolated from the E15 dorsal telencephalon, MACS sorted for A2B5⁺/PSA-NCAM⁻ cells and expanded for 7 days in bFGF. Cultures were then switched to differentiation conditions and labeled after 6-9 days (depending on condition) with markers that identified differentiated progeny. As show in FIGS. 4A, C and D, cells were capable of generating GalC⁺ oligodendrocytes in PDGF-AA plus T3/T4 and GFAP⁺ astrocytes in 1% FBS, but were unable to generate neurons in NT3 and RA. To exclude the possibility that the failure of neuronal generation from the A2B5⁺/PSA-NCAM⁻/beta III tubulin⁻ was due to an inadequate pro-neuronal environment, freshly isolated, non-selected cells from E15 dorsal telencephala were cultured at clonal density in the presence of NT3 and RA for 6 days and labeled clones with anti-beta III tubulin. As shown in FIG. 5A, clones possessing the ability to generate neurons in the pro-neuron condition were readily identifiable, indicating the pro-neuronal condition used was adequate to elicit neuron formation from a competent cell.

In accordance with the generation of oligodendrocytes from spinal cord derived GRP cells, an O4⁺ intermediate cell type was seen upon exposure to PDGF-AA plus T3/T4 for 4 days (FIG. 4B). BMP-4, shown previously to increase astroglial cell commitment and implicated in the switch from neuron to astrocyte formation in the telencephalon was unable to generate GFAP⁺ cells until 10 days after the onset of BMP exposure (FIG. 4E), but did induce expression of the known GRP derived astrocyte precursor cell marker, CD44, after 6 days in vitro (FIG. 4F). Taken together, the results presented confirmed that the A2B5⁺/PSA-NCAM⁻ dorsal telencephalic cell population is capable of generating oligodendrocytes and astrocytes but not neurons.

The A2B5⁺/PSA-NCAM⁻ Population Generates Similar Numbers of Clones Containing Oligodendrocytes or Astrocytes, but No Clones Containing Neurons.

While the initial in vitro differentiation experiments indicated the restriction of the A2B5⁺/PSA-NCAM⁻ population to the glial lineage, a distinction between the presence of a bipotential cell that can generate oligodendrocytes and astrocytes and the presence of a heterogeneous population of APCs and OPCs was necessary. To distinguish between these two possibilities, A2B5⁺/PSA-NCAM⁻ cells grown in culture for one week were passaged and re-plated at clonal density. Clones were then exposed to bFGF (proliferative), PDGF-AA plus T3T4 (pro-oligodendrocyte), 1% FBS (pro-astrocyte), or NT3 plus RA (pro-neuron) in order to determine the differentiation potential of individual clones. A clone was considered to be capable of generating the specified cell types by the presence of at least one oligodendrocyte per clone, at least one astrocyte per clone, or at least one neuron per clone, in the respective condition.

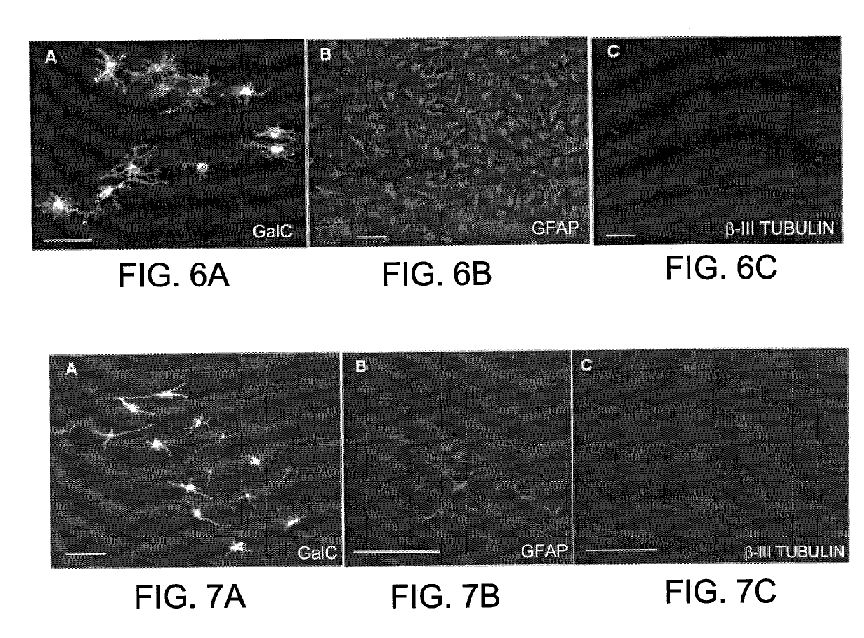

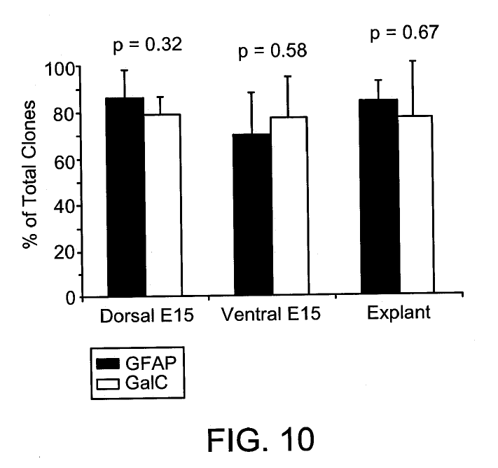

A2B5⁺/PSA-NCAM⁻ cells from the dorsal telencephalon gave rise to clones capable of generating oligodendrocytes (FIG. 6A), astrocytes (FIG. 6B) but not neurons (FIG. 6C) after six days of exposure to the differentiation conditions. In four independent experiments, a total of 223 clones exposed to PDGF-AA plus T3/T4, a total of 164 clones exposed to 1% FBS, and more than 200 clones exposed to NT3 plus RA were analyzed. 79% of the clones exposed to PDGF-AA plus T3/T4 contained at least one GalC⁺ oligodendrocyte, 87% of all clones exposed to 1% serum (115 clones) contained at least one GFAP⁺ astrocyte, while none of the clones exposed to NT3 plus RA contained a neuron. A summary of the GFAP⁺ and GalC⁺ clones is presented in FIG. 10, and indicates a similar percentage of astrocyte-containing clones and oligodendrocyte-containing clones in the respective conditions, a result consistent with a cell capable of generating both oligodendrocytes and astrocytes.

The Splitting of A2B5⁺/PSA-NCAM⁻ Clones Reveals the Potential to Generate Oligodendrocytes and Astrocytes from a Single Founder Cell

The analysis of the clonal data demonstrate that the A2B5⁺/PSA-NCAM⁻ population comprised a cell capable of generating both oligodendrocytes and astrocytes when exposed to appropriate conditions in parallel wells. As the presently known conditions that are required to induce cell differentiation along a specific lineage do not allow the generation of oligodendrocytes and astrocytes in a single clone at the same time, an alternative method was needed to determine whether the progeny arising from a single A2B5⁺/PSA-NCAM⁻ cell was able to generate oligodendrocytes and astrocytes. “Clone-splitting” analysis was initiated, as outlined in FIG. 3C. The cells were plated at clonal density in 100 mm dishes and allowed to propagate in bFGF (10 ng/ml) until a clone size of approximately 200 cells was achieved. Clones were selected based on the presence of cells consistent with the bipolar morphology of precursor cells. Each selected clone was passaged and re-plated amongst four wells of a 24 well plate and exposed to the previously used differentiating conditions. Clones passaged in this manner gave rise to oligodendrocytes in PDGF-AA plus T3T4 (FIG. 7A), astrocytes in 1% FBS (FIG. 7B) but did not generate neurons in NT3 and RA (FIG. 7C) after 6 days of exposure to the indicated conditions. Each split clone was capable of generating oligodendrocytes and astrocytes but not neurons in the respective conditions, confirming the potential of the initial A2B5⁺/PSA-NCAM⁻ founder cell to generate both oligodendrocytes and astrocytes, and allowing for its classification as a glial restricted precursor cell.

Dorsal Glial Restricted Precursor Cells are Generated De Novo from the Dorsal Telencephalon

In order to determine if the dorsal telencephalon is competent to generate glial restricted precursor cells de novo, or is a result of ventral cell infiltration, E12.5 dorsal telencephalon was mechanically separated from the ventral telencephalon and the dorsal explant was grown for 2 days in vitro. The physical separation of the dorsal telencephalon from the ventral telencephalon allowed for the simulated development of the dorsal telencephalon in the absence of ventral cell types until a time period comparable to an E15 dorsal telencephalon. As E12.5 is prior to the known entrance of ventral cells into the dorsal telencephalon, any cells present or generated in the two day culture period were decisively of dorsal origin.

Explants were harvested after two days of in vitro growth in Neural Basal Media in the absence of bFGF. This was important to minimize the possibility that the culture conditions would lead to a “ventralization” of the explants, although no such an effect was observed in vitro when dissociated cells were cultured in the presence of bFGF.

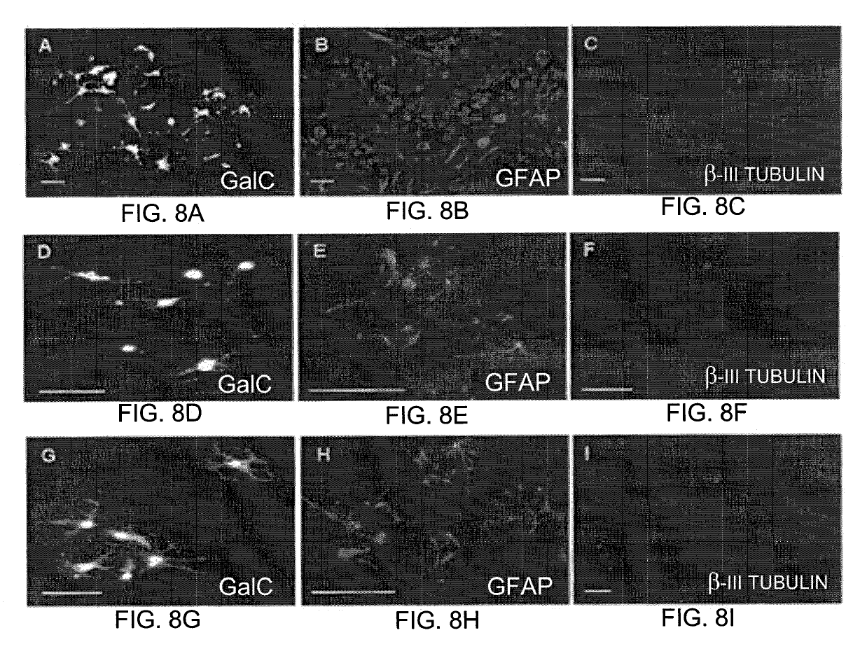

Explant tissue was cultured for 2 days, after which A2B5⁺/PSA-NCAM⁻ cells were selected by MACS separation from the dissociated explants and cultured for an additional 7 days before being subjected to mass culture differentiation and clonal analyses. Mass culture studies indicated that the explant-derived A2B5⁺/PSA-NCAM⁻ cell population possessed similar in vitro differentiation abilities as the glial restricted precursor population from the dorsal telencephalon. Explant cells were induced to generate GalC⁺ oligodendrocytes with PDGF-AA plus T3/T4 (FIG. 8A), GFAP⁺ astrocytes with 1% FBS (FIG. 8B), and did not generate neurons in NT3 plus RA (FIG. 8C). The explant derived A2B5⁺/PSA-NCAM⁻ cells grown at clonal density gave rise to 145 out of 190 (76%) clones containing at least one GalC⁺ oligodendrocyte when exposed to PDGF-AA plus T3/T4 (FIG. 8D). 144 out of 173 (84%) clones contained at least one astrocyte when exposed to 1% FBS (FIG. 8E), and clones containing at least one neuron when exposed to NT3 and RA could not be detected (FIG. 8F). A summary of the clones generated by the dorsal explant A2B5⁺/PSA-NCAM⁻ cell population is provided (FIG. 10).

To further the characterization of the explant derived putative glial restricted precursor population, A2B5⁺/PSA-NCAM⁻ cells isolated from 2 day in vitro grown explants were plated at clonal density and the differentiation potential of the clonal progeny was characterized as outlined in FIG. 3C. Six clones were selectively passaged and the cells from each clone were divided among four wells of a 24 well plate for exposure to the differentiation conditions. Cells from the split clones were able to generate GalC⁺ oligodendrocytes in PDGF-AA plus T3/T4 (FIG. 8G), GFAP⁺ astrocytes in 1% FBS (FIG. 8H), but were unable to generate neurons in NT3 and RA (FIG. 8I). These data confirm the ability of the dorsal telencephalon to give rise to an A2B5⁺/PSA-NCAM⁻ glial restricted precursor population independent of cellular migration from ventral regions and indicates a potential dorsal origin for the telencephalic glial restricted precursor population in vivo.

A Ventral Glial Restricted Precursor Cell can be Isolated from the E15 Rat Telencephalon

As no ventral telencephalic cell from the developing telencephalon has been reported to be able to give rise to astrocytes and oligodendrocytes but not neurons, the analysis was expended to determine whether a glial restricted precursor cell also exists in the ventral aspect of the early telencephalon.

Due to the multiple origins of OPC generation, analysis of a putative ventral glial restricted precursor population was begun by dissecting the medial ganglionic eminence (MGE) and the anterior entopeduncular area (AEP) of E15 ventral telencephala. Pdgf-alpha expression studies indicated OPC presence in these areas. The potential problem of isolating a heterogeneous population of glial restricted precursor cells and OPCs was addressed by growing freshly isolated A2B5⁺/PSA-NCAM⁻ cells in the presence of 10 ng/ml PDGF. This condition has been previously shown to maintain OPCs but unable to support GRP cell survival. Surviving cells grown in this manner were beta III tubulin⁺ and few if any A2B5⁺ cells were detected. Taken together, the absence of a PDGF responsive A2B5⁺ population and the known inability of OPCs to generate type-I astrocytes (A2B5⁻/GFAP⁺) allowed for the selective determination of a ventral glial restricted population.

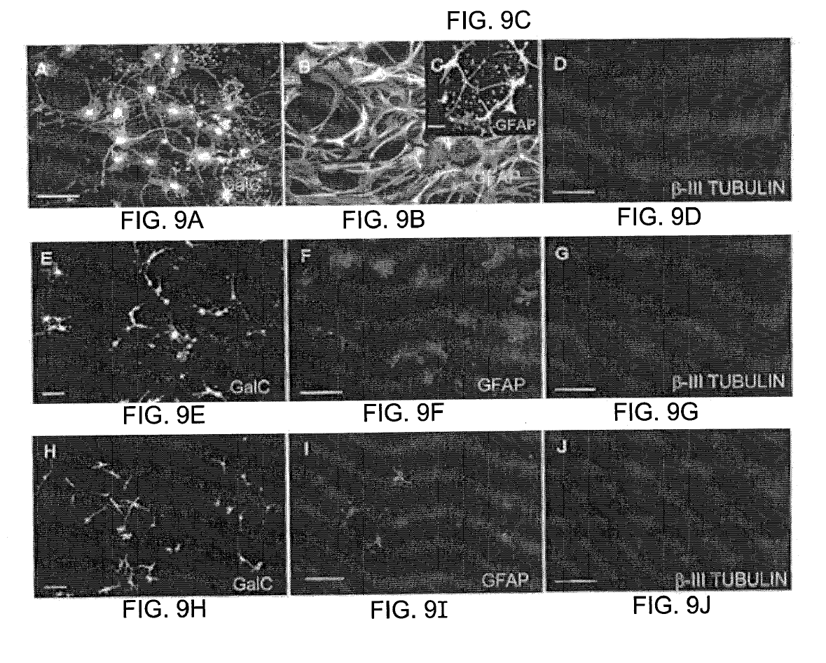

A2B5⁺/PSA-NCAM⁻ cells were isolated and characterized in vitro using the same experimental approaches described before and summarized in FIG. 3. Mass culture studies confirmed the ability of this ventral A2B5⁺/PSA-NCAM⁻ cell population to generate GalC⁺ oligodendrocytes in PDGF-AA plus T3/T4 (FIG. 9A), GFAP⁺ astrocytes in 1% FBS (FIG. 9B) and the inability to generate neurons in NT3 and RA (FIG. 9D). Clonal analysis established the capacity of individual A2B5⁺/PSA-NCAM⁻ cells to generate 174 out of 223 (78%) total clones counted containing at least one GalC⁺ oligodendrocytes in PDGF plus T3T4 (FIG. 9E), 115 clones out of 164 (70%) total clones counted containing at least one GFAP⁺ astrocytes (FIG. 9F), but an inability to generate clones containing at least one neuron in NT3 and RA (FIG. 9G). A summary of the clones counted is provided in FIG. 10. In order to confirm the effectiveness of NT3 and RA to induce a neuronal cell fate, freshly isolated unselected ventral telencephalic cells were plated at clonal density. Unselected cells from the ventral telencephalon possessing the necessary differentiation potential generated beta III tubulin⁺ cell clones identifiable after 6 days of exposure to NT3 plus RA (FIG. 5B).

A2B5⁺/GFAP⁺ cells were not detected in 1% FBS or with exposure to ciliary neurotrophic factor (CNTF; FIG. 9C), a condition known to induce A2B5⁺/GFAP⁺ Type-II astrocytes from spinal cord derived GRPs. Type-II astrocyte generation and oligodendrocyte generation is presently thought to be the differentiation profile of the OPC, while the ability to generate both Type-I (A2B5⁻/GFAP⁺) and Type-II (A2B5⁺/GFAP⁺) astrocytes and GalC⁺ oligodendrocytes from a restricted glial precursor is characteristic only of the GRP cell.

For further in vitro characterization, freshly isolated ventral A2B5⁺/PSA-NCAM⁻ cells were plated at clonal density and selectively passaged and split as outlined in FIG. 3C. The cells from a single divided clone generated GalC⁺ oligodendrocytes in PDGF-AA plus T3/T4 (FIG. 9H), GFAP⁺ astrocytes in 1% FBS (FIG. 9I) but did not generate neurons in NT3 plus RA (FIG. 9J). These results confirm glial restricted precursor cells are present in the E15 ventral telencephalon.

In Vivo Production of Myelinating Oligodendrocytes and Astrocytes by Telencephalic Glial Restricted Precursor Cells

The in vitro analyses identified the existence of dorsal and ventral A2B5⁺/PSA-NCAM⁻ glial restricted precursor populations in the E15 telencephalon capable of generating oligodendrocytes and/or astrocytes but unable to generate neurons under conditions that generally promote neuronal lineage. Data also indicated that the dorsal telencephalon possesses the potential to generate the A2B5⁺/PSA-NCAM⁻ glial restricted precursor population without the presence of ventral cell components.

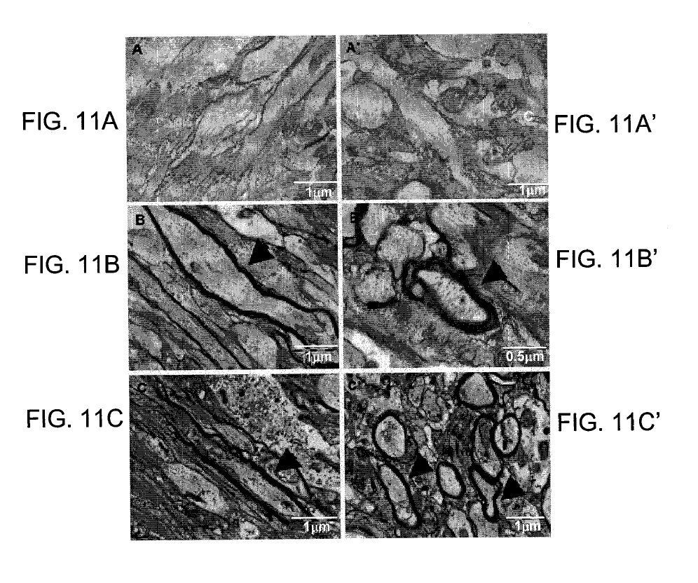

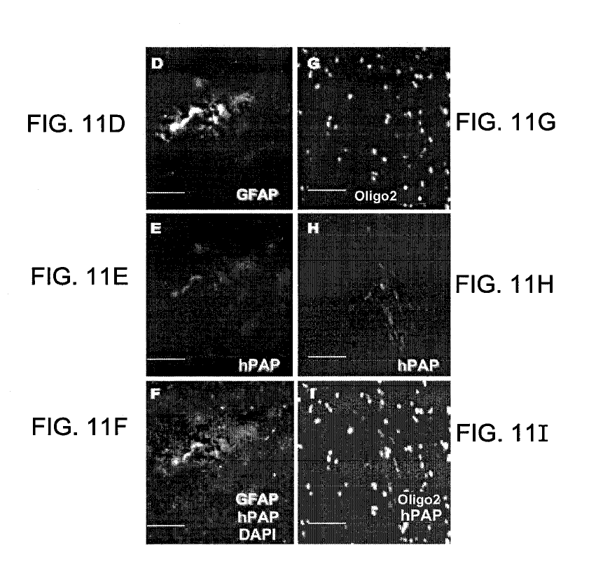

A2B5⁺/PSA-NCAM⁻ glial restricted precursor cells were isolated from 1) the E15 dorsal telencephalon and 2) E12.5 dorsal telencephalic explants grown in vitro for two days for transplantation into the forebrain of postnatal shiverer mice. The shiverer mouse contains a deletion in the MBP gene resulting in little to no compacted myelin formation. This animal provided an avenue for examining the ability of the dorsal glial restricted precursor population to generate functional oligodendrocytes that, importantly, can contribute to the myelin composition of the forebrain. The dorsal and explant derived glial restricted precursor populations were transplanted into the subcortical region of the left hemisphere of postnatal day 18 homozygous shiverer mice. The contralateral hemisphere of each mouse was not injected and served as the control for basal myelin presence and appearance. At three weeks post-transplantation, animals were perfused and 1.5 mm coronal sections were prepared for electron microscopy. EM images taken of the non-injected hemispheres showed thin, non-compacted myelin sheets, typical of shiverer forebrains, in longitudinally sectioned (FIG. 11A) and cross-sectioned (FIG. 11A′) axonal fibers present in the coronal sections. EM images of the hemisphere containing the transplanted E15 dorsal glial restricted precursor population showed numerous dense, compacted myelinated fibers in the subcortical white matter, seen in longitudinally sectioned fibers (FIG. 11B) and cross-sectioned fibers (FIG. 11B′), extending from the site of injection to more lateral aspects of the dorsal forebrain. Longitudinal and cross-sections of dense, compacted myelinated fibers were readily identifiable in EM images acquired from coronal sections of the hemisphere containing the transplanted explant derived glial restricted precursor population as well (FIGS. 11C and C′).

One hallmark of the spinal cord derived GRP cell that distinguishes this cell from an OPC is its ability to produce astrocytes upon transplantation. In order to determine the in vivo astrocytic potential of the dorsal and ventral telencephalic glial restricted precursor cells, isolated glial restricted precursor populations from E15 telencephala of transgenic rat embryos expressing human placental alkaline phosphatase (hPAP) were transplanted into the forebrains of PO Sprague Dawley rat pups, a time point coinciding with peak astrocyte formation and the beginning of dorsal born oligodendrocyte precursors. At postnatal day 10, pups were sacrificed and sections were analyzed for co-localization of hPAP and GFAP. Double positive cells could be found throughout the transplanted regions of host brains receiving dorsal (FIG. 11D-F) glial restricted precursors, although regions showing hPAP cells not co-localizing with GFAP were also seen. Olig2⁺/hPAP⁺ cells could also be visualized in the transplanted regions, indicating the presence of oligodendrocyte precursors (O2As) and/or oligodendrocytes (FIG. 11G-I). These transplantation studies confirmed the ability of the dorsal glial restricted precursor population to generate myelinating oligodendrocytes, as well as the ability of the dorsal glial restricted precursor population to generate astrocytes and cells of the oligodendrocyte lineage upon transplantation.

Two A2B5⁺/PSA-NCAM⁻ cell populations were identified: one isolated from the E15 dorsal telencephalon and the other isolated from the E15 ventral telencephalon. The designation of cells as GRP, OPC or NSC can include the analysis of the cell type-specific differentiation potential (for review, see Noble et al 2006). While it can be expected that NSC can generate oligodendrocytes, astrocytes and neurons, lineage restricted cells do not display the full array of cell types upon differentiation.

The mass culture analyses, clonal analyses, clone splitting analyses, and in vivo transplantation experiments of the A2B5⁺/PSA-NCAM⁻/beta III tubulin⁻ telencephalic cell population demonstrated their ability to generate cells of the glial lineage but an inability to differentiate into neurons. This differentiation profile strongly resembles that of the previously described GRP population of the E13.5 spinal cord. In addition to the similar differentiation profile, the telencephalic glial restricted precursor populations are, like the spinal cord GRP population, responsive to bFGF as a mitogen and survival factor and can also be isolated from both dorsal and ventral aspects of the respective tissues. The data also establishes the capability of the dorsal telencephalon to generate a telencephalic glial restricted precursor population in the absence of ventral cell tissue.

There were, however, detectable differences between the telencephalic cells and the previously studied spinal cord cells, including the astrocyte generation upon exposure to BMP-4, as well as a lack of Type-II astrocyte generation in response to CNTF. The last characteristic, in particular, makes a distinction between this telencephalic precursor cell population and the extensively studied OPCs isolated from postnatal rat brains.

The identification of tGRPs also offers a defined source for astrocytes. It has been shown in the spinal cord that astrocytes occur in both dorsal and ventral regions, and a subset of astrocytes and oligodendrocytes arises from cells of ventral origin migrating to and residing in the dorsolateral subventricular zone. Astrocytic populations have also been identified in other regions of the developing telencephalon, but the source of these cells has remained elusive. tGRPs that arise both ventrally and dorsally can account for the generation of at least a subset of astrocytes in the developing telencephalon.

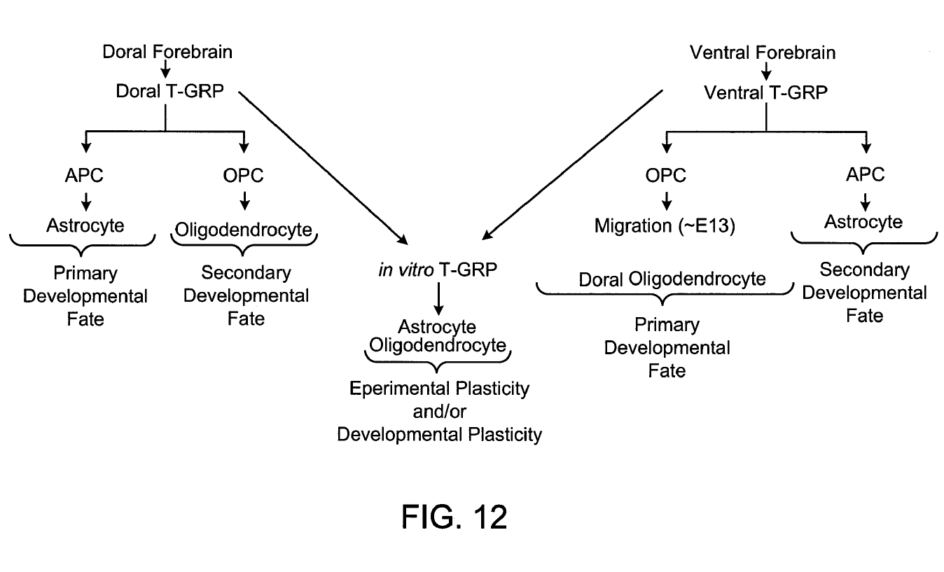

The identification of tGRPs allows for the unification of the various existing models of glial origin, and to this end the following model for gliogenesis in the telencephalon if shown (FIG. 12). The data show that at least two tGRP populations are generated independently in the ventral and dorsal aspect of the embryonic telencephalon. The dorsal tGRP population is developmentally fated towards APC and astrocyte generation early in development, while the ventral tGRP population shows an initial developmental fate towards OPC generation due to environmental signals. Removal of environmental cues (e.g. BMP dorsally and Shh ventrally) by isolation and in vitro culture allows for the emergence of the developmental plasticity of each population, as seen with the generation of astrocytes and oligodendrocytes from ventral and dorsal tGRPs, respectively.

Later in development, as signals change or are modified to provide a permissive environment for glial cell maturation, this model affords the potential of each tGRP population to contribute to the generation of an alternate glial cell type, revealing the secondary developmental fate of each tGRP population. Importantly, the isolation of a prototypical tGRP population from either the ventral or dorsal regions, regardless of the time point, provides a cell population capable of generating both oligodendrocytes and astrocytes, but not neurons.

### Example 2

A. Astrocytes Derived from tGRP Using CNTF are Distinct from Astrocytes Derived from scGRPs.

The transplantation of spinal cord derived GDA^{sgp130} (glial restricted precursor cells induced to differentiate into astrocytes using signaling molecules that act through the gp130) or undifferentiated GRP cells resulted in robust neuropathic pain. Forepaw withdrawal thresholds to a mechanical stimulus and the withdrawal response latency of any paw from a heat source were measured before and after dorsolateral funiculus transection. GDA^{gp130} transplanted animals showed a significant increase in sensitivity to both mechanical and heat stimuli by 2 weeks post injury, an effect that intensified between the second and third weeks and persisted through 5 weeks post injury, the last time point tested. Animals that received intra-injury transplants of undifferentiated GRP cells also developed increased sensitivity to both mechanical and heat stimuli, although with a delayed time course to that shown by GDA^{gp130} transplanted animals. GRP transplanted animals began to show increased sensitivity in both tests by 3 weeks post injury/transplantation, a sensitivity that also persisted through 5 weeks post injury. In contrast, transplantation of astrocyte generated from GRP cells via induction using BMP (GDA^{BMP}) did not show any increased sensitivity to mechanical or heat stimuli at any time point up to 5 weeks post injury compared to pre-injury responses (2 Way Repeated Measures ANOVA p>0.05) a result in striking contrast with the effects of transplantations of GDA^{sgp130} or GRP cells.

Independent studies showed that one of the major differences of GDA^{BMP} and GDA^{gp130} is their expression of the transcription factor Olig2. GDA^{BMP} express GFAP but are not Olig2+. In contrast, GDA^{gp130} co-express GFAP and Olig2. In light of these data, the expression of Olig2 was characterized in astrocytes derived from tGRPs.

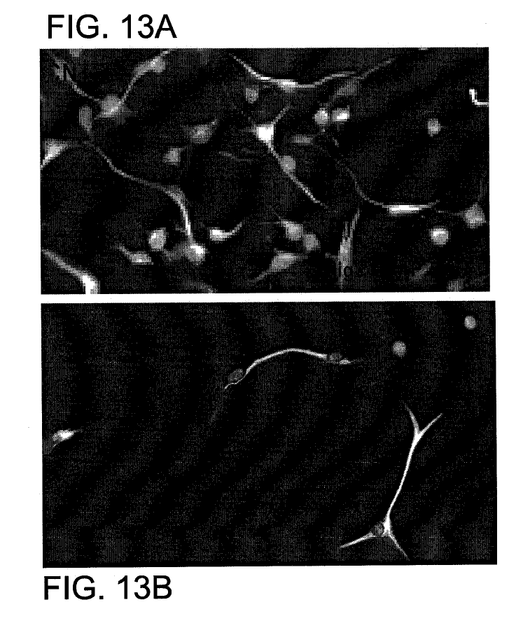

As shown in FIG. 13, tGRP cells induced with CNTF do not express Olig2 and are hence distinct from scGRP derived GDA^{gp130}.

B. tGRPs Derived from the Dorsal Versus the Ventral Telencephalon have Distinct Redox Status.

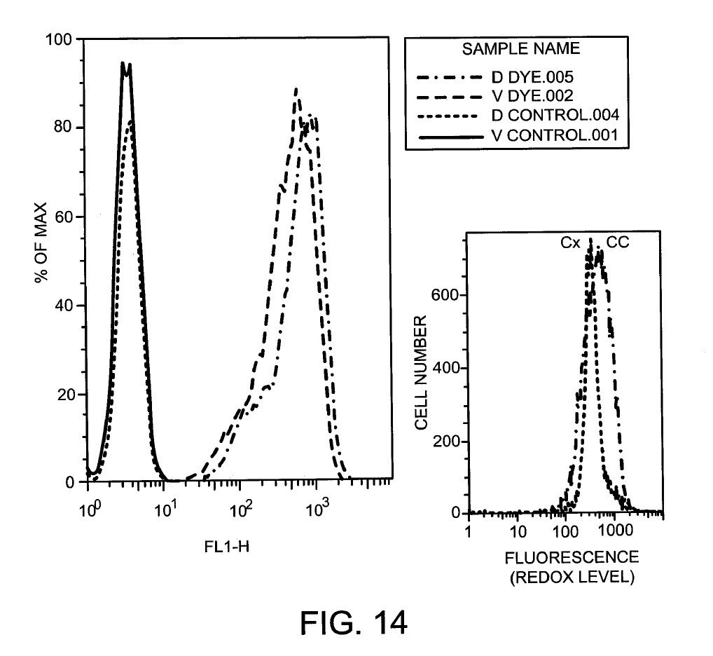

Intracellular redox status of dorsal and ventral tGRPs was assayed using Dihydrocalcein (DHC), a cell permeable fluorescent measure of intracellular oxidases. dtGRPs were found to be more oxidized than vtGRPs (FIG. 14). As a comparison, the intracellular redox status of OPCs from corpus callosum (CC) and cortex (Cx) were included as a comparison.

C. Intermediate Generation of Oligodendrocytes from tGRPs

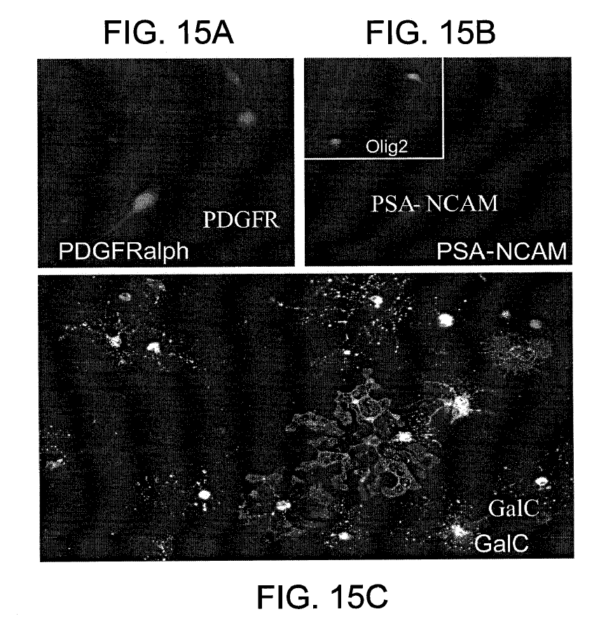

Previously, tGRPs were shown to generate GalC+oligodendrocytes. Further investigation has expanded this characterization and indicates tGRPs generate GalC+oligodendrocytes via a PSA-NCAM/PDGFRalpha/Olig2+ intermediate (FIG. 15). This intermediate cell, generated from tGRP cultures by removing bFGF and adding PDGF, is distinguishable from the tGRP, shown previously to be negative for PSA-NCAM, PDGFRalpha and Olig2.

1. A population of isolated telencephalic glial restricted precursor (tGRP) cells.

2. The population of claim 1, wherein the tGRP cells are isolated from the dorsal telencephalon.

3. The population of claim 2, wherein the tGRP cells are isolated from the ventral telencephalon.

4. A composition comprising the population of claim 1 and a culture medium or pharmaceutical carrier.

5. A method of treating a CNS injury in a subject, comprising administering to the subject a composition comprising isolated tGRP cells.

6. The method of claim 5, wherein the CNS injury is caused by a demylenating disease.

7. The method of claim 5, wherein the CNS injury is caused by trauma or stroke.

8. A method of increasing gliogenesis in a subject, comprising administering to the subject a composition comprising isolated tGRP cells.

9. The method of claim 8, wherein the tGRP cells are isolated from the dorsal telencephalon.

10. The method of claim 8, wherein the tGRP cells are isolated from the ventral telencephalon.

11. A population of isolated Olig2⁻ glial restricted (GRP) cells.

12. The population of claim 11, wherein the Olig2⁻ GRP cells are tGRP cells.

13. The population of claim 12, wherein the tGRP cells are isolated from the dorsal telencephalon.

14. A composition comprising the population of claim 11 and a culture medium or pharmaceutical carrier.

15. A composition comprising the population of claim 13 and a culture medium or pharmaceutical carrier.

16. A method of treating a CNS injury in a subject, comprising administering to the subject a composition comprising isolated Olig2⁻ GRP cells.

17. The method of claim 16, wherein the Olig2⁻ GRP cells are tGRP cells.

18. The population of claim 17, wherein the tGRP cells are isolated from the dorsal telencephalon.

19. A method of treating a CNS injury in a subject, comprising:
(a) isolating a population of tGRP cells;
(b) deriving a GFAP⁺ cell or population of GFAP⁺ cells from the isolated population of tGRP cells; and
(c) administering to the subject a composition comprising the derived GFAP⁺ cell, or one or more derived GFAP⁺ cell of the derived GFAP⁺ cell population.

20. The method of claim 19, wherein the GFAP⁺ cell or population of GFAP⁺ cells derived from the isolated population of tGRP cells comprises an astrocyte progenitor cell (APC).

21. The method of claim 19, wherein the GFAP⁺ cell or population of GFAP⁺ cells derived from the isolated population of tGRP cells comprises a GDA cell.

22. The method of claim 19, wherein the GFAP⁺ cell or population of GFAP⁺ cells derived from the isolated population of tGRP cells comprises a type-1 astrocyte.

23. The method of claim 19, wherein the tGRP cells are isolated from the dorsal telencephalon.

24. A method of treating a CNS injury in a subject, comprising:
(a) isolating a population of tGRP cells;
(b) deriving a GalC⁺ cell or population of GalC⁺ cells from the isolated population of tGRP cells; and
(c) administering to the subject a composition comprising the derived GalC⁺ cell, or one or more derived GalC⁺ cell of the GalC⁺ cell population.

25. The method of claim 24, wherein the GalC⁺ cell or population of GalC⁺ cells derived from the isolated population of tGRP cells comprise an oligodendrocyte.
